(19) United States
(12) Patent Application Publication
(10) Pub. No.: US 2015/0203899 A1
(43) Pub. Date: Jul. 23, 2015

(54) DIAGNOSIS OF ACTIVE TUBERCULOSIS BY DETERMINING THE MRNA EXPRESSION LEVELS OF MARKER GENES IN BLOOD

(71) Applicant: IMPERIAL INNOVATIONS LIMITED, London (GB)

(72) Inventors: Michael Levin, London (GB); Lachlan Coin, London (GB); Robert Wilkinson, London (GB); Neil French, London (GB)

(21) Appl. No.: 14/418,270

(22) PCT Filed: Jul. 29, 2013
(86) PCT No.: PCT/EP2013/065887

§ 371 Date: Jan. 29, 2015

(30) Foreign Application Priority Data
Jul. 31, 2012 (GB) 1213636.2

(51) Int. Cl. C12Q 1/68; C12Q 1/70

(52) U.S. Cl. CPC C12Q 1/689; C12Q 1/703; C12Q 2600/16; C12Q 2600/158

(57) ABSTRACT

The present disclosure relates to a method of distinguishing active TB in the presence of a complicating factor, for example, latent TB and/or co-morbidities, such as those that present similar symptoms to TB, such as HIV. The method employs a 27 gene signature to distinguish active tuberculosis from latent TB infection, a 44 gene signature to distinguish active TB from other diseases such as HIV and/or a 53 gene signature to discriminate active TB from latent TB and other diseases. The disclosure also relates to a gene signature employed in the method, a bespoke gene chip for use in the method and a disease risk score obtainable from the method.

The present disclosure relates to a method of distinguishing active TB in the presence of a complicating factor, for example, latent TB and/or co-morbidities, such as those that present similar symptoms to TB. The disclosure also relates to a gene signature employed in the said method and to a bespoke gene chip for use in the method. The disclosure further relates to use of known gene chips in the methods of the disclosure and kits comprising the elements required for performing the method. The disclosure also relates to use of the method to provide a composite expression score which can be used in the diagnosis of TB, particularly in a low resource setting.

### BACKGROUND

An estimated 8.8 million new cases and 1.45 million deaths are caused by Tuberculosis, TB (short for tubercle bacillus) each year (World Health Organisation statistics 2011). TB is an infectious disease caused by various species of mycobacteria, typically Mycobacterium tuberculosis. Tuberculosis usually attacks the lungs but can also affect other parts of the body. It is spread through the air when people who have an active TB infection cough, sneeze, or otherwise transmit their saliva. Most infections in humans result in an asymptomatic, latent infection, and about one in ten latent infections eventually progress to active disease, which, if left untreated, kills more than 50% of those infected. Immunosuppression and malnutrition are among the risk factors for developing active TB.

The classic symptoms are a chronic cough with blood-tinged sputum, fever, night sweats, and weight loss (the latter giving rise to the formerly prevalent colloquial term “consumption”). Infection of organs other than the lungs causes a wide range of symptoms. Treatment is difficult and requires long courses of multiple antibiotics. Antibiotic resistance is a growing problem with numbers of multi-drug-resistant tuberculosis cases on the rise. This is, in part, due to the length of treatment needed. Those infected with latent TB are typically asymptomatic and therefore either forget or decided not to take antibiotics. Those infected with active TB often cease treatment when the symptoms clear even though the infection remains.

Correct diagnosis is of utmost importance in the treatment of TB. The treatment regimens for active TB and latent TB are different and so it is important to diagnose the two conditions correctly in order to provide appropriate therapy.

Diagnosis of TB is particularly complicated as it cannot solely be based on symptoms. This is for two reasons: those infected with latent TB exhibit no symptoms and active TB may present similar symptoms to other infections or illnesses. Matters may be further complicated by the fact that TB may not be the only infection or illness that the patient has. Co-morbidities and co-infections often mask the symptoms of active TB and thus the latter goes undiagnosed and untreated. If active TB goes untreated the patient has a high probability of death due to the disease. Not only does TB present similar symptoms to other infectious or non-infectious conditions but it also presents similar radiological features. Thus identifying the presence of TB definitively can be difficult.

Diagnosis is therefore multi-facetted, relying on clinical and radiological features (commonly chest X-rays), sputum microscopy (with or without culture), tuberculin skin test (TST), blood tests, as well as microscopic examination and microbiological culture of bodily fluids. In many places, such as Africa, which often do not have the resources needed to make a full diagnosis, this is a major impediment to tuberculosis treatment and control. Culture facilities are largely unavailable for TB diagnosis in most African hospitals.

All of the known methods of diagnosis have drawbacks, particularly in HIV co-infected persons in whom radiological features are often atypical:
Sputum microscopy often has low sensitivity in HIV infected patients with TB because cavitatory lung disease is less common in this group, resulting in sputum negative microscopy (Schultz 2010).
Tuberculin skin testing (TST) and Interferon Gamma Release Assays (IGRA) do not discriminate TB from latent TB infection (LTBI) and are of limited utility in African countries where LTBI is highly prevalent in the healthy population. In 2010 Metcalfe et al concluded that neither TST nor IGRA have value for active tuberculosis diagnosis in the context of HIV co-infection in low and middle income countries.
Although molecular diagnosis has improved detection of M. tuberculosis DNA in sputum, the sensitivity of this approach is lower in smear negative samples, even if culture positive, and the method does not detect solely extra-pulmonary disease.

Consequently, a high proportion of active TB cases in sub-Saharan Africa remain undiagnosed, and post-mortem studies show TB to be a frequent, undiagnosed cause of death. There is an urgent need for improved diagnostic tests for TB, particularly in patients co-infected with HIV.

RNA expression analysis by microarray has emerged as a powerful tool for understanding disease biology. Many diseases, including cancer and infectious diseases are associated with specific transcriptional profiles in blood or tissue.

In an influential study, Berry et al (2001) found a 393 transcript signature derived in a UK cohort that was able to distinguish TB from LTBI, and an 86 transcript signature able to distinguish TB from other inflammatory diseases. However, these signatures were derived from UK populations of HIV-uninfected individuals. Therefore these signatures are of limited application in Africa, where HIV infection and LTBI are endemic.

Many previous TB diagnostic biomarker studies have focused on distinguishing patients with TB from healthy uninfected or LTBI (Maertzdorf et al 2011a 2011b, Jacobsen et al 2007) or have used other disease controls which are not representative of the real world clinical diseases from which TB needs to be distinguished in Africa (Maertzdorf et al 2012, Berry et al 2010). Furthermore, previous studies have excluded HIV co-infected patients who are in fact the group in which new diagnostics are most needed.

Thus there is a need to identify biomarkers that discriminate TB from other diseases prevalent in African populations, where the burden of the HIV/TB pandemic is greatest.

### SUMMARY OF THE INVENTION

The present disclosure provides a method for detecting active TB in a subject derived sample in the presence of a complicating factor, comprising the step of detecting the modulation of at least 60% of the genes in a signature selected from the group consisting of:
a) a 27 gene signature shown in Table 3,
b) a 44 gene signature shown in Table 4,
c) a 53 gene signature shown in Table 5,
d) a combination of signatures a) and b), a) and c), b) and c) or a) and b) and c).

Advantageously use of the appropriate signature in a method according to the present disclosure allows the robust and accurate identification of the presence of active TB or the differentiation of active TB from latent TB in the most relevant clinical setting, for example Africa. The detection is not prevented by co-morbidity in the patient, such as HIV or malaria. This is a huge step forward on the road to treating TB because it allows accurate diagnosis which, in turn, allows patients to be appropriately treated. Furthermore, the components for use in the method to detect active TB can be provided in a simple format for use in low resource and/or rural settings.

In another aspect of the disclosure there is provided a gene chip comprising one or more of the gene signatures selected from the group consisting of:
a) 60 to 100% of a 27 gene signature shown in Table 3,
b) 60 to 100% of a 44 gene signature shown in Table 4,
c) 60 to 100% of a 53 gene signature shown in Table 5,
d) a combination of signatures a) and b), a) and c), b) and c) or a) and b) and c), and
e) optionally one or more house-keeping genes.

In a further aspect the present disclosure includes use of a known or commercially available gene chip in the method of the present disclosure.

Advantageously the different expression patterns represented by the gene signatures employed in the method of the present disclosure correlate across geographic location and HIV infected status (i.e. positive or negative). That is to say, the method is applicable to different geographic locations regardless of the presence or absence of HIV.

In a further aspect the present disclosure provides the treatment of active TB or latent TB after diagnosis employing the method herein.

### BRIEF DESCRIPTION OF THE FIGURES

FIGS. 1A and B. Study overview showing patient numbers and analysis. HIV−=HIV-uninfected, HIV+=HIV infected, TB=active tuberculosis, LTBI=latent TB infection, OD=other diseases (see Table 1B).

FIG. 2. Clustering of training (A/B) and test (C/D) cohorts using transcripts identified by elastic net for TB vs. LTBI (A/C) and TB vs. OD (B/D) (training set comprised A: n_{TB}=157 n_{LTBI}=128 and B: n_{TB}=153 n_{OD}=140. Test set comprised C: n_{TB}=37 n_{LTBI}=39 and D n_{TB}=42 n_{OD}=34).

The rows are transcripts (red=up-regulated, green=down-regulated). Columns are cases regardless of HIV status (purple are TB cases, green are LTBI, light blue are OD).

FIG. 2A. Clustering of TB vs. non-TB (i.e. LTBI and OD) based on the TB/non-TB 53 transcript signature applied to the South African and Malawi training (A) and test (B) cohorts. Patients are represented as columns (light grey=active TB, dark grey=LTBI and OD) and individual transcripts are shown in rows (light grey=up-regulated, dark grey=down-regulated).

FIG. 3. Disease risk score and Receiver Operator Curves based on the TB vs. LTBI 27 transcript signature (shown in A, B and C) and the TB vs. OD 44 transcript signature (shown in D, E and F) applied to the South African (SA)/Malawi HIV+/− test cohort (A/D) (n_{TB}=37 n_{LTBI}=39/n_{TB}=42 n_{OD}=34) and independent validation cohorts (Berry et al 2010) comprising UK patients (B/E) (n_{TB}=21 n_{LTBI}=21 n_{OD}=82) and South African patients (C/F) (n_{TB}=20 n_{LTBI}=31 n_{OD}=82). Sensitivity, specificity are reported in Table 2B.

HIV+=HIV-infected, HIV−=HIV-uninfected

FIG. 4A. Diagnostic criteria for inclusion as either a TB case or as a latent TB infected case.

Definite TB case: a participant with a clinical condition consistent with tuberculosis and microbiological confirmation with evidence from at least two specimens confirming the presence of Acid Fast Bacilli (AFB) with at least one specimen confirmed on culture as MTB complex.

Latent TB infected case: a participant who is clinically assessed as healthy and not suffering from a clinical syndrome in which tuberculosis is likely. The individuals will have a tuberculin skin test (TST) size of 10 mm or more if HIV negative, or 5 mm or more if HIV positive and a positive Interferon Gamma Release Assay (IGRA) and negative sputum culture. Sputums were only collected in Malawi if cough was productive, when at least two samples would be collected. LTBI criteria were later relaxed to allow a positive TST and/or a positive IGRA to facilitate recruitment in Malawi. This change was made prior to any RNA expression measurements.

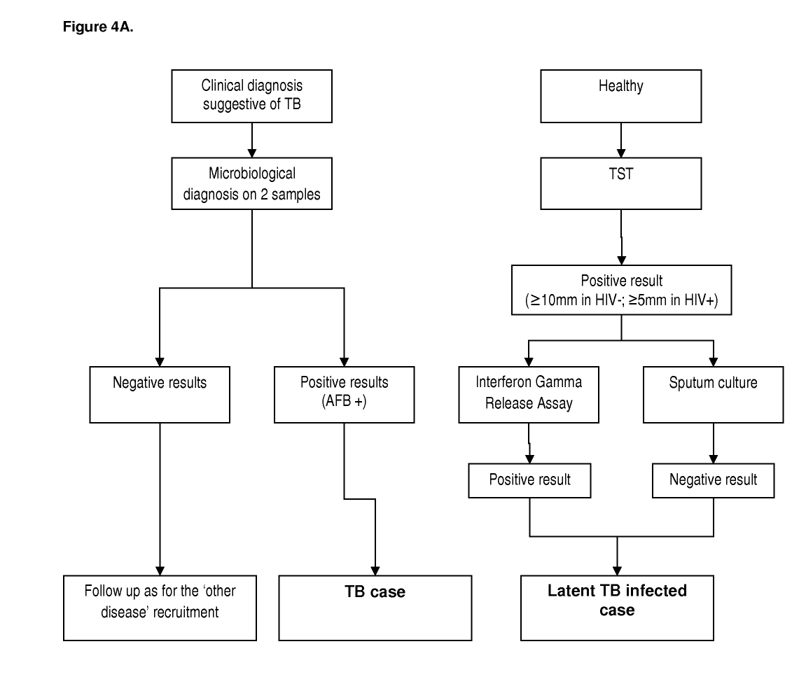

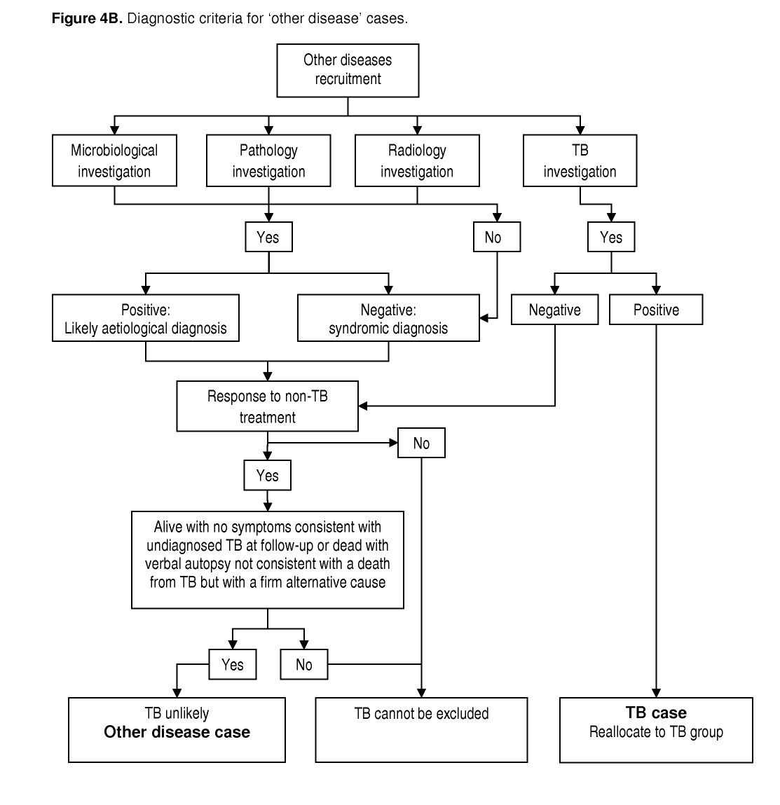

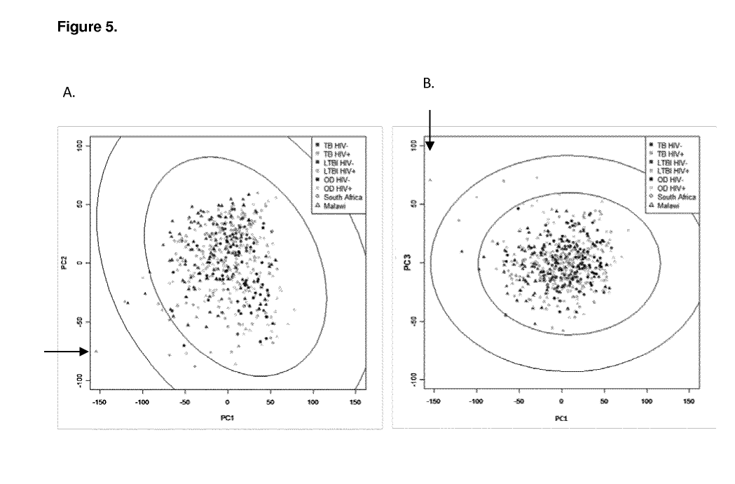

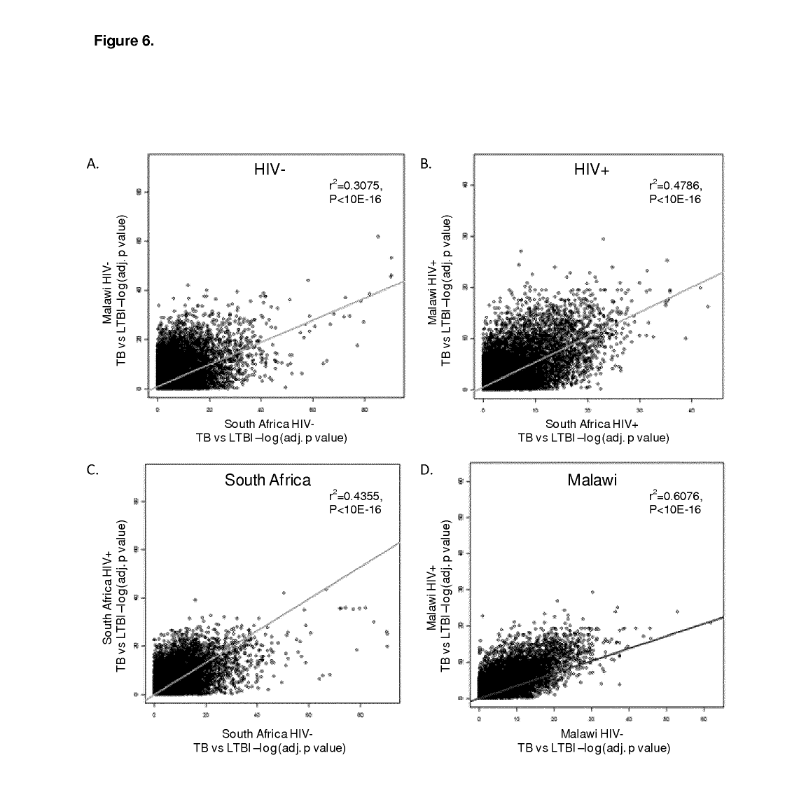

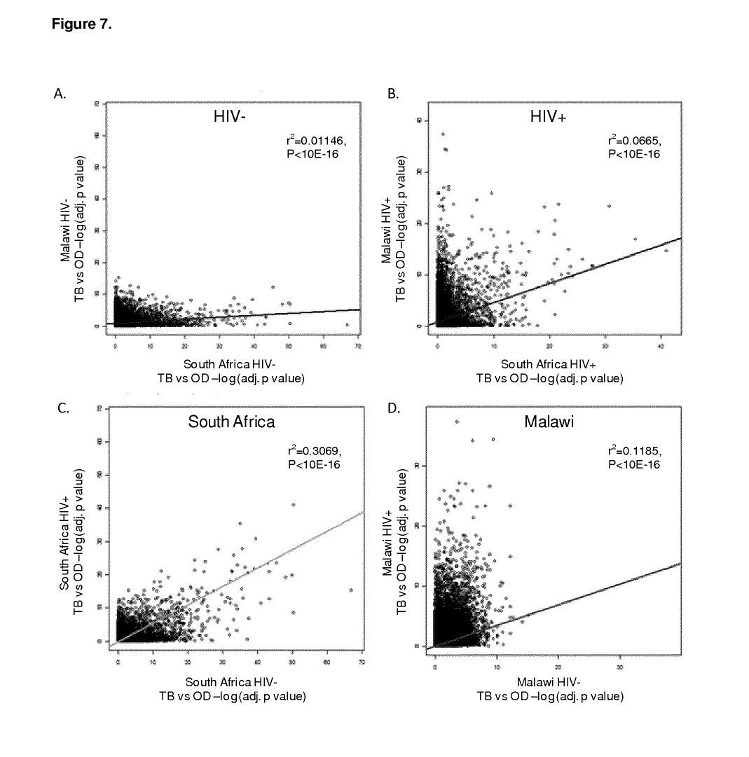

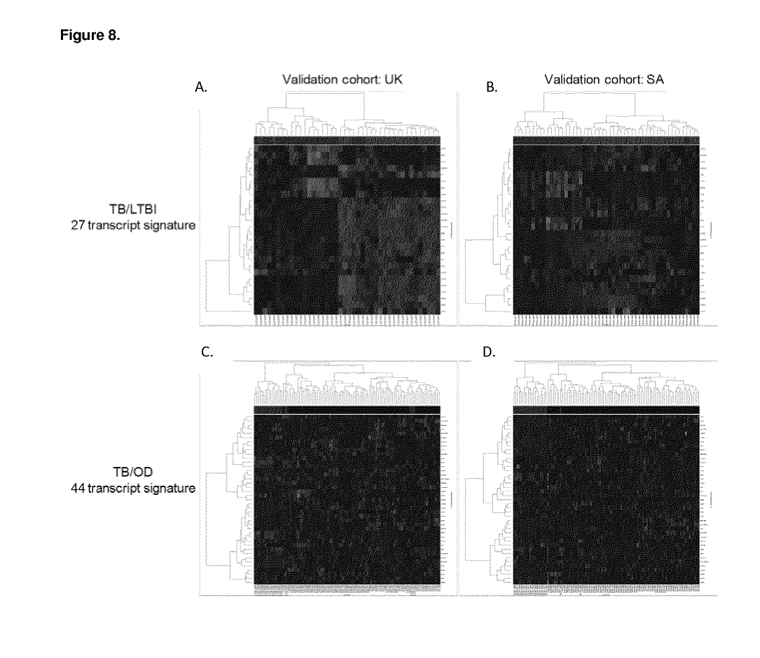

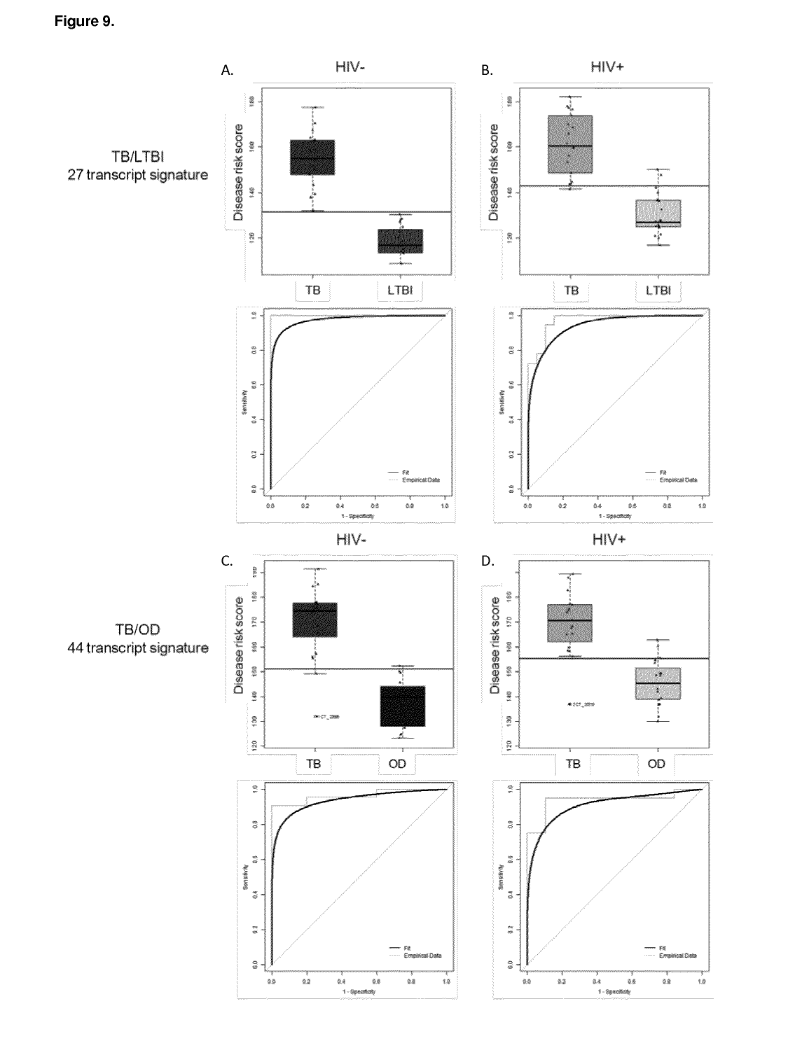

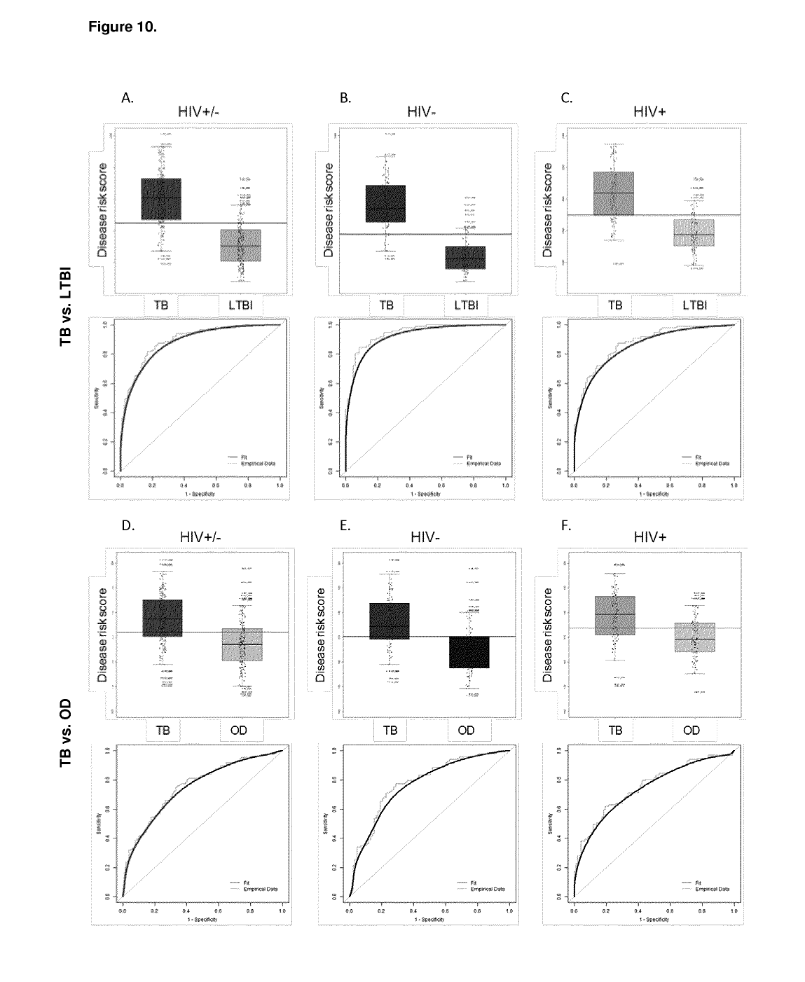

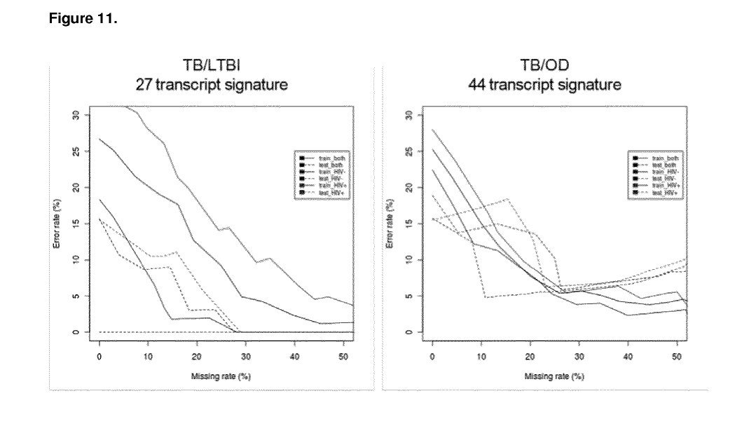

FIG. 4B. Diagnostic criteria for ‘other disease’ cases.

Other disease case: A participant with a disease syndrome that on presentation includes tuberculosis in the differential diagnosis, but following clinical management will have tuberculosis excluded and a firm alternative diagnosis established.

FIG. 5. Principal components analysis (PCA) of the microarrayed samples. PCA plot based on all the genes on all the samples after background adjustment and normalisation. A) shows PCA1 & PCA2 and B) shows PCA1 & PCA3. The sample highlighted (categorised as active TB HIV+ from Malawi) was removed from the analysis. Rings are levels of confidence (0.9 inner circle, 0.9999 outer circle).

FIG. 6. Concordance of differential expression by location of cohort (A/B) and by HIV status (C/D) for the active TB vs. latent TB infection cohorts in South Africa (SA) and Malawi. Negative logarithm of the corrected p-values in TB vs. LTBI between SA and Malawi for HIV-uninfected (HIV−) cohort (A) and HIV-infected (HIV+) cohort (B); and between HIV-uninfected and -infected cohorts in SA (C) and in Malawi (D). There were positive correlations between all comparisons. p=0.05 is equivalent to −log p value=1.3.

FIG. 7. Concordance of differential expression by location of cohort (A/B) and by HIV status (C/D) for the active TB vs. other disease cohorts in South Africa (SA) and Malawi. Negative logarithm of the corrected p-values in TB vs. OD between SA and Malawi for HIV-uninfected (HIV−) cohort (A) and HIV-infected (HIV+) cohort (B); and between HIV-uninfected and -infected cohorts in SA (C) and in Malawi (D). There were positive correlations between all comparisons. Note, the correlation between SA/Malawi HIV− cohorts is less than in SA/Malawi HIV+ cohorts which may reflect the different spectra of conditions in the ‘other disease’ cohorts. p=0.05 is equivalent to −log p value=1.3.

FIG. 8. Clustering of TB vs. LTBI based on the TB vs. LTBI 27 transcript signature (A/B) and TB vs. OD 44 transcript signature (C/D) applied to independent UK (A/C) and South African validation cohorts (B/D) of Berry et al (2010). Patients are represented as columns (red=TB, green=LTBI, Blue=other diseases) and individual transcripts are shown in rows (red=up-regulated, green=down-regulated).

FIG. 9. Disease risk score and Receiver Operator Curves (ROC) based on the TB vs. LTBI 27 transcript signature (A/B) and the TB vs. OD 44 transcript signature (C/D) applied to the HIV-uninfected (HIV−) (A/C) and HIV-infected (HIV+) (B/D) test cohort. Area Under Curve (AUC), sensitivities and specificities are reported in Table 2A.

FIG. 10. Disease risk score and ROC based on transcript signatures of Berry et al (2010) for TB vs. LTBI (A/B/C) and TB vs. OD (D/E/F) applied to the combined training and test cohorts in both HIV-uninfected (HIV−) and HIV-infected (HIV+) (A/D), HIV−(B/E) and HIV+(C/F) cohorts. See Table 2B for sensitivities, specificities and AUC. The Berry et al signature does not differentiate TB in the presence of other disease.

FIG. 11. Shows the error rate of classification in relation to the percentage of misclassified cases for the 27 gene signature and the 44 gene signature.

For coloured versions of the figures refer to Kaforou et al (PLOS medicine—submitted 2013)

### DETAILED DESCRIPTION

In one embodiment there is detected the modulation of at least 60% of the genes in a signature such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% providing the signature retains the ability to detect/discriminate the relevant clinical status without significant loss of specificity and/or sensitivity. The details of the gene signatures are given below.

In one embodiment the exact gene list in one or more of Tables 2, 3 and 4 is employed.

In one embodiment of the present disclosure the gene signature is the minimum set of genes required to optimally detect the infection or discriminate the disease.

Optimally is intended to mean the smallest set of genes needed to detect active TB without significant loss of specificity and/or sensitivity of the signature's ability to detect or discriminate.

Detect or detecting as employed herein is intended to refer to the process of identifying an active TB infection in a sample, in particular through detecting modulation of the relevant genes in the signature.

Discriminate refers to the ability of the signature to differentiate between different disease status, for example latent and active TB. Detect and discriminate are interchangeable in the context of the gene signature.

In one embodiment the method is able to detect an active TB infection in a sample.

Subject as employed herein is a human suspected of TB infection from whom a sample is derived. The term patient may be used interchangeably although in one embodiment a patient has a morbidity.

Modulation of gene expression as employed herein means up-regulation or down-regulation of a gene or genes.

Up-regulated as employed herein is intended to refer to a gene transcript which is expressed at higher levels in a diseased or infected patient sample relative to, for example, a control sample free from a relevant disease or infection, or in a sample with latent disease or infection or a different stage of the disease or infection, as appropriate.

Down-regulated as employed herein is intended to refer to a gene transcript which is expressed at lower levels in a diseased or infected patient sample relative to, for example, a control sample free from a relevant disease or infection or in a sample with latent disease or infection or a different stage of the disease or infection.

The modulation is measured by measuring levels of gene expression by an appropriate technique.

Gene expression as employed herein is the process by which information from a gene is used in the synthesis of a functional gene product. These products are often proteins, but in non-protein coding genes such as ribosomal RNA (rRNA), transfer RNA (tRNA) or small nuclear RNA (snRNA) genes, the product is a functional RNA. That is to say, RNA with a function.

A complicating factor as employed herein refers to at least one clinical status or at least one medical condition that would generally render it more difficult to identify the presence of active TB in the sample, for example a latent TB infection or a co-morbidity.

Co-morbidity as employed herein refers the presence of one or more disorders or diseases in addition to TB, for example malignancy such as cancer or co-infection. Co-morbidity may or may not be endemic in the general population.

In one embodiment the co-morbidity is a co-infection.

Co-infection as employed herein refers to bacterial infection, viral infection such as HIV, fungal infection and/or parasitic infection such as malaria. HIV infection as employed herein also extends to include AIDS.

In one embodiment other disease (OD) is a co-morbidity.

In one embodiment the 44 gene signature is able to detect active TB in the presence of a co-morbidity such as a co-infection. This is despite the increased inflammatory response of the patient to said other infection.

In one embodiment co-morbidity is selected from malignancy, HIV, malaria, pneumonia, Lower Respiratory Tract Infection, Pneumocystis Jirovecii Pneumonia, pelvic inflammatory disease, Urinary Tract Infection, bacterial or viral meningitis, hepatobiliary disease, cryptococcal meningitis, non-TB pleural effusion, empyema, gastroenteritis, peritonitis, gastric ulcer and gastritis.

In one embodiment malignancy is a neoplasia, such as bronchial carcinoma, lymphoma, cervical carcinoma ovarian carcinoma, mesothelioma, gastric carcinoma, metastatic carcinoma, benign salivary tumour, dermatological tumour or Kaposi's sarcoma.

In one embodiment there is provided a method for detecting active TB in a subject derived sample in the presence of a complicating factor, comprising the step of detecting the modulation of at least 60% of the genes in a signature selected from the group consisting of:
a) a 27 gene signature shown in Table 3,
b) a 44 gene signature shown in Table 4,
c) a combination of signatures a) and b).

The 27 gene signature shown in Table 3 is useful in discriminating active TB infection from latent TB infection.

Active TB as employed herein refers to a person who is infected with TB which is not latent.

In one embodiment active TB is where the disease is progressing as opposed to where the disease is latent.

In one embodiment a person with active TB is capable of spreading the infection to others.

In one embodiment a person with active TB has one or more of the following: a skin test or blood test result indicating TB infection, an abnormal chest x-ray, a positive sputum smear or culture, active TB bacteria in his/her body, feels sick and may have symptoms such as coughing, fever, and weight loss.

In one embodiment a person with active TB has one or more of the following symptoms: coughing, bloody sputum, fever and/or weight loss.

In one embodiment the active TB infection is pulmonary and/or extra-pulmonary.

Pulmonary as employed herein refers to an infection in the lungs.

Extra-pulmonary as employed herein refers to infection outside the lungs, for example, infection in the pleura, infection in the lymphatic system, infection in the central nervous system, infection in the genito-urinary tract, infection in the bones, infection in the brain and/or infection in the kidneys.

Symptoms of pulmonary TB include: a persistent cough that brings up thick phlegm, which may be bloody; breathlessness, which is usually mild to begin with and gradually gets worse; weight loss; lack of appetite; a high temperature of 38° C. (100.4° F.) or above; extreme tiredness; and a sense of feeling unwell.

Symptoms of lymph node TB include: persistent, painless swelling of the lymph nodes, which usually affects nodes in the neck, but swelling can occur in nodes throughout your body; over time, the swollen nodes can begin to release a discharge of fluid through the skin.

Symptoms of skeletal TB include: bone pain; curving of the affected bone or joint; loss of movement or feeling in the affected bone or joint and weakened bone that may fracture easily.

Symptoms of gastrointestinal TB include: abdominal pain; diarrhoea and anal bleeding.

Symptoms of genitourinary TB include: a burning sensation when urinating; blood in the urine; a frequent urge to pass urine during the night and groin pain.

Symptoms of central nervous system TB include: headaches; being sick; stiff neck; changes in your mental state, such as confusion; blurred vision and fits.

Latent TB as employed herein refers to a subject who is infected with TB but is asymptomatic. A sputum test will generally be negative and the infection cannot be spread to others.

In one embodiment a person with latent TB infection has one of more of the following: a skin test or blood test result indicating TB infection, a normal chest x-ray and a negative sputum test, TB bacteria in his/her body that are alive, but inactive, does not feel sick, cannot spread TB bacteria to others

In one embodiment a person with latent TB needs treatment to prevent TB disease becoming active.

In one embodiment the method of the present disclosure is able to differentiate TB from different conditions/diseases or infections which have similar clinical symptoms.

Similar symptoms as employed herein includes one or more symptoms from pulmonary TB, lymph node TB, skeletal TB, gastrointestinal TB, genitourinary TB and/or central nervous system TB.

In one embodiment the method according to the present disclosure is performed on a subject with acute infection.

In a further embodiment the sample is a subject sample from a febrile subject, that is to say with a temperature above the normal body temperature of 37.5° C.

Thus in one embodiment DNA or RNA from the subject sample is analysed.

In one embodiment the sample is solid or fluid, for example blood or serum or a processed form of any one of the same.

A fluid sample as employed herein refers to liquids originating from inside the bodies of living people. They include fluids that are excreted or secreted from the body as well as body water that normally is not. Includes amniotic fluid, aqueous humour and vitreous humour, bile, blood serum, breast milk, cerebrospinal fluid, cerumen (earwax), chyle, endolymph and perilymph, gastric juice, mucus (including nasal drainage and phlegm), sputum, peritoneal fluid, pleural fluid, saliva, sebum (skin oil), semen, sweat, tears, vaginal secretion, vomit, urine. Particularly blood and serum.

Blood as employed herein refers to whole blood, that is serum, blood cells and clotting factors, typically peripheral whole blood.

Serum as employed herein refers to the component of whole blood that is not blood cells or clotting factors. It is plasma with fibrinogens removed.

In one embodiment the subject derived sample is a blood sample.

In one or more embodiments the analysis is ex vivo.

Ex vivo as employed herein means that which takes place outside the body.

In one embodiment one or more, for example 1 to 21, such as 2, 3, 4, 5, 6, 7, 8, 9, 10, 11, 12, 13, 14, 15, 16, 17, 18, 19 or 20, genes are replaced by a gene with an equivalent function provided the signature retains the ability to detect/discriminate the relevant clinical status without significant loss in specificity and/or sensitivity.

In one embodiment the genes employed have identity with genes listed in the relevant tables.

In one embodiment the 27 gene signature comprises or consists of at least up-regulated genes CD79A, CD79B, CXCR5, GNG7, CCR6, ZNF296.

In one embodiment the 27 gene signature comprises or consists of at least down-regulated genes C5, FAM20A, DUSP3, GAS6, S100A8, FCGR1B, LHFPL2, FCGR1A, MPO, FCGR1C, GAS6, C1QB, ANKRD22, FCGR1B, GBP6, C4ORF18, C1QC, FLVCR2, VAMP5, SMARCD3, and LOC728744.

In one embodiment the 27 gene signature comprises or consists of at least up-regulated genes and optionally down-regulated genes C5, FAM20A, DUSP3, GAS6, S100A8, FCGR1B, LHFPL2, FCGR1A, MPO, FCGR1C, GAS6, C1QB, ANKRD22, FCGR1B, GBP6, C4ORF18, C1QC, FLVCR2, VAMP5, SMARCD3, and LOC728744.

In one embodiment the 44 gene signature comprises or consists of at least up-regulated genes ARG1, IMPA2, RP5-1022P6.2, ORM1, EBF1, PDK4, MAK, VPREB3, HS.131087, MAP7, TMCC1, HS.162734, MAP7, and PGA5.

In one embodiment the 44 gene signature comprises or consists of at least down-regulated genes HM13BTN3A1, UGP2, CYB561, GBP6, CYB561, DUSP3, LOC196752, ALDH1A1, PRDM1, CERKL, HM13, RNF19A, MIR1974, PPPDE2, GJA9, CREB5, SERPING1, LOC389386, SEPT_{—}4, RBM12B, CALML4, LHFPL2, CASC1, C19ORF12, HLA-DPB1, CD74, ALDH1A1, AAK1, and LOC100133800.

In one embodiment the 44 gene signature comprises or consists of at least up-regulated genes ARG1, IMPA2, RP5-1022P6.2, ORM1, EBF1, PDK4, MAK, VPREB3, HS.131087, MAP7, TMCC1, HS.162734, MAP7, PGA5 and optionally down-regulated genes HM13BTN3A1, UGP2, CYB561, GBP6, CYB561, DUSP3, LOC196752, ALDH1A1, PRDM1, CERKL, HM13, RNF19A, MIR1974, PPPDE2, GJA9, CREB5, SERPING1, LOC389386, SEPT_{—}4, RBM12B, CALML4, LHFPL2, CASC1, C19ORF12, HLA-DPB1, CD74, ALDH1A1, AAK1, and LOC100133800.

In one embodiment the 53 gene signature comprises or consists of at least up-regulated genes GNG7, BLK, OSBPL10, CXCR5, HEY1, COL9A2, SPIB, LOC90925, ILMN_{—}1916292, EBF1, VPREB3, TMCC1, MAP7, PGA5, and ILMN_{—}1893697.

In one embodiment the 53 gene signature comprises or consists of at least down-regulated genes UGP2, BTN3A1, DUSP3, GBP6, CALML4, FZD2, CYB561, LHFPL2, CYB561, CASC1, RNU4ATAC, VPS13B, PPPDE2, ALDH1A1, GBP5, GAS6, SEP_{—}4, FCGR1B, POLB, CREB5, SIGLEC11, LOC389386, DEFA1B, LOC650546, FAM26F, FCGR1A, DEFA1B, ALDH1A1, ANKRD22, IF127L2, DEFA1, MIR21, DEFA3, FCGR1C, UHMK1, CD74, IL15, and CREG1.

In one embodiment the 53 gene signature comprises or consists of at least up-regulated genes GNG7, BLK, OSBPL10, CXCR5, HEY1, COL9A2, SPIB, LOC90925, ILMN_{—}1916292, EBF1, VPREB3, TMCC1, MAP7, PGA5, ILMN_{—}1893697 and optionally down-regulated genes UGP2, BTN3A1, DUSP3, GBP6, CALML4, FZD2, CYB561, LHFPL2, CYB561, CASC1, RNU4ATAC, VPS13B, PPPDE2, ALDH1A1, GBP5, GAS6, SEP_{—}4, FCGR1B, POLB, CREB5, SIGLEC11, LOC389386, DEFA1B, LOC650546, FAM26F, FCGR1A, DEFA1B, ALDH1A1, ANKRD22, IF127L2, DEFA1, MIR21, DEFA3, FCGR1C, UHMK1, CD74, IL15, and CREG1.

In one embodiment the 27 and 44 gene signatures are tested in parallel.

In one embodiment the 27 and 53 gene signatures are tested in parallel.

In one embodiment the 44 and 53 gene signatures are tested in parallel.

In one embodiment the 27, 44 and 53 gene signatures are tested in parallel.

In one embodiment each of the genes in the 27, 44 and 53 gene signatures is significantly differentially expressed in the sample with active TB compared to a comparator group.

Significantly differentially expressed as employed herein means the sample with active TB shows a log 2 fold change >0.5.

In the 27 gene signature the comparator group is LTBI.

In the 44 gene signature the comparator group is a person with “other disease” (OD), that is a disease that is not active TB but has similar symptoms.

In the 53 gene signature group the comparator group is LTBI+OD. Thus the 53 gene signature is suitable for identifying active TB in the presence of any other complicating factor.

“Presented in the form of” as employed herein refers to the laying down of genes from one or more of the signatures in the form of probes on a microarray.

Accurately and robustly as employed herein refers to the fact that the method can be employed in a practical setting, such as Africa, and that the results of performing the method properly give a high level of confidence that a true result is obtained.

High confidence is provided by the method when it provides few results that are false positives (i.e. the result suggests that the subject has active TB when they do not) and also has few false negatives (i.e. the result suggest that the subject does not have active TB when they do).

High confidence would include 90% or greater confidence, such as 91, 92, 93, 94, 95, 96, 97, 98, 99 or 100% confidence when an appropriate statistical test is employed.

In one embodiment the method provides a sensitivity of 80% or greater such as 90% or greater in particular 95% or greater, for example where the sensitivity is calculated as below:

$$\begin{matrix}sensitivity = \frac{number of true positives}{number of true positives + number of false negatives} \\ = probability of a positive test given that the patient is ill\end{matrix}$$

In one embodiment the method provides a high level of specificity, for example 80% or greater such as 90% or greater in particular 95% or greater, for example where specificity is calculated as shown below:

$$\begin{matrix}specificity = \frac{number of true nega tives}{number of true nega tives + number of false posi tives} \\ = probability of a nega tive test given that the patient is we ll\end{matrix}$$

In one embodiment the sensitivity of method of the 27 gene signature is 83 to 100%, such as 84, 85, 86, 87, 88, 89, 90, 91, 92, 93, 94, 95, 96, 97, 98 or 99%.

In one embodiment the specificity of the method of the 27 gene signature is 75 to 100%, such as 76, 77, 78, 79, 80, 81, 82, 83, 84, 85, 86, 87, 88, 89, 90, 91, 92, 93, 94, 95, 96, 97, 98 or 99%.

In one embodiment the sensitivity of the method of the 44 gene signature is 77 to 100%, such as 78, 79, 80, 81, 82, 83, 84, 85, 86, 87, 88, 89, 90, 91, 92, 93, 94, 95, 96, 97, 98 or 99%.

In one embodiment the specificity of the method of the 44 gene signature is 68 to 100%, such as 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99%.

There are a number of ways in which gene expression can be measured including microarrays, tiling arrays, DNA or RNA arrays for example on gene chips, RNA-seq and serial analysis of gene expression.

Any suitable method of measuring gene modulation may be employed in the method of the present disclosure.

In one embodiment the gene expression data is generated from a microarray, such as a gene chip.

Microarray as employed herein includes RNA or DNA arrays, such as RNA arrays.

A gene chip is essentially a microarray that is to say an array of discrete regions, typically nucleic acids, which are separate from one another and are, for example arrayed at a density of between, about 100/cm² to 1000/cm², but can be arrayed at greater densities such as 10000/cm².

The principle of a microarray experiment, is that mRNA from a given cell line or tissue is used to generate a labelled sample typically labelled cDNA or cRNA, termed the ‘target’, which is hybridised in parallel to a large number of, nucleic acid sequences, typically DNA or RNA sequences, immobilised on a solid surface in an ordered array. Tens of thousands of transcript species can be detected and quantified simultaneously. Although many different microarray systems have been developed the most commonly used systems today can be divided into two groups.

Using this technique, arrays consisting of more than 30,000 cDNAs can be fitted onto the surface of a conventional microscope slide. For oligonucleotide arrays, short 20-25mers are synthesised in situ, either by photolithography onto silicon wafers (high-density-oligonucleotide arrays from Affymetrix) or by ink-jet technology (developed by Rosetta Inpharmatics and licensed to Agilent Technologies).

Alternatively, pre-synthesised oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of pre-synthesised longer oligonucleotides (50-100mers) has recently been developed to counteract these disadvantages.

In one embodiment the gene chip is an off the shelf, commercially available chip, for example HumanHT-12 v4 Expression BeadChip Kit, available from Illumina, NimbleGen microarrays from Roche, Agilent, Eppendorf and Genechips from Affymetrix such as HU-UI 33.Plus 2.0 gene chips.

In an alternate embodiment the gene chip employed in the present invention is a bespoke gene chip, that is to say the chip contains only the target genes which are relevant to the desired profile. Custom made chips can be purchased from companies such as Roche, Affymetrix and the like. In yet a further embodiment the bespoke gene chip comprises a minimal disease specific transcript set.

In one embodiment the chip comprises or consists of 60-100% of the 27 genes listed in Table 3.

In one embodiment the chip comprises or consists of 60-100% of the 44 genes listed in Table 4.

In one embodiment the chip comprises or consists of 60-100% of the 53 genes listed in Table 5.

In one embodiment the chip comprises or consists of 60-100% of the 27 genes listed in Table 3 in combination with 60-100% of the 44 genes listed in Table 4.

In one embodiment the chip comprises or consists of 60-100% of the 27 genes listed in Table 3 in combination with 60-100% of the 53 genes listed in Table 5.

In one embodiment the chip comprises or consists of 60-100% of the 44 genes listed in Table 4 in combination with 60-100% of the 53 genes listed in Table 5.

In one embodiment the chip comprises or consists of 60-100% of the 27 genes listed in Table 3 in combination with 60-100% of the 44 genes listed in Table 4 and 60-100% of the 53 genes listed in Table 5.

In one or more embodiments above the chip may further include 1 or more, such as 1 to 10, house-keeping genes.

In one embodiment the gene expression data is generated in solution using appropriate probes for the relevant genes.

Probe as employed herein is intended to refer to a hybridisation probe which is a fragment of DNA or RNA of variable length (usually 100-1000 bases long) which is used in DNA or RNA samples to detect the presence of nucleotide sequences (the DNA target) that are complementary to the sequence in the probe. The probe thereby hybridises to single-stranded nucleic acid (DNA or RNA) whose base sequence allows probe-target base pairing due to complementarity between the probe and target.

In one embodiment the method according to the present disclosure and for example chips employed therein may comprise one or more house-keeping genes. House-keeping genes as employed herein is intended to refer to genes that are not directly relevant to the profile for identifying the disease or infection but are useful for statistical purposes and/or quality control purposes, for example they may assist with normalising the data, in particular a house-keeping gene is a constitutive gene i.e. one that is transcribed at a relatively constant level. The housekeeping gene's products are typically needed for maintenance of the cell. Examples include actin, GAPDH and ubiquitin.

In one embodiment minimal disease specific transcript set as employed herein means the minimum number of genes need to robustly identify the target disease state.

Minimal discriminatory gene set is interchangeable with minimal disease specific transcript set.

Normalising as employed herein is intended to refer to statistically accounting for background noise by comparison of data to control data, such as the level of fluorescence of house-keeping genes, for example fluorescent scanned data may be normalized using RMA to allow comparisons between individual chips. Irizarry et al 2003 describes this method.

Scaling as employed herein refers to boosting the contribution of specific genes which are expressed at low levels or have a high fold change but still relatively low fluorescence such that their contribution to the diagnostic signature is increased.

Fold change is often used in analysis of gene expression data in microarray and RNA-Seq experiments, for measuring change in the expression level of a gene and is calculated simply as the ratio of the final value to the initial value i.e. if the initial value is A and final value is B, the fold change is B/A. Tusher et al 2001.

In programs such as Arrayminer, fold change of gene expression can be calculated. The statistical value attached to the fold change is calculated and is the more significant in genes where the level of expression is less variable between subjects in different groups and, for example where the difference between groups is larger.

In one embodiment the subject is an adult. Adult is defined herein as a person of 18 years of age or older.

In one embodiment the subject is a child. Child as employed herein refers to a person under the age of 18, such as 5 to 17 years of age.

The step of obtaining a suitable sample from the subject is a routine technique, which involves taking a blood sample. This process presents little risk to donors and does not need to be performed by a doctor but can be performed by appropriately trained support staff. In one embodiment the sample derived from the subject is approximately 2.5 ml of blood, however smaller volumes can be used for example 0.5-1 ml.

Blood or other tissue fluids are immediately placed in an RNA stabilizing buffer such as included in the Pax gene tubes, or Tempus tubes.

If storage is required then it should usually be frozen within 3 hours of collections at −80° C.

In one embodiment the gene expression data is generated from RNA levels in the sample.

For microarray analysis the blood may be processed using a suitable product, such as PAX gene blood RNA extraction kits (Qiagen).

Total RNA may also be purified using the Tripure method—Tripure extraction (Roche Cat. No. 1 667 165). The manufacturer's protocols may be followed. This purification may then be followed by the use of an RNeasy Mini kit—clean-up protocol with DNAse treatment (Qiagen Cat. No. 74106).

Quantification of RNA may be completed using optical density at 260 nm and Quant-IT RiboGreen RNA assay kit (Invitrogen—Molecular probes RI 1490). The Quality of the 28s and 18s ribosomal RNA peaks can be assessed by use of the Agilent bioanalyser.

In another embodiment the method further comprises the step of amplifying the RNA. Amplification may be performed using a suitable kit, for example TotalPrep RNA Amplification kits (Applied Biosystems).

In one embodiment an amplification method may be used in conjunction with the labelling of the RNA for microarray analysis. The Nugen 3′ ovation biotin kit (Cat: 2300-12, 2300-60).

The RNA derived from the subject sample is then hybridised to the relevant probes, for example which may be located on a chip. After hybridisation and washing, where appropriate, analysis with an appropriate instrument is performed.

In performing an analysis to ascertain whether a subject presents a gene signature indicative of disease or infection according to the present disclosure, the following steps are performed: obtain mRNA from the sample and prepare nucleic acids targets, hybridise to the array under appropriate conditions, typically as suggested by the manufactures of the microarray (suitably stringent hybridisation conditions such as 3×SSC, 0.1% SDS, at 50<0>C) to bind corresponding probes on the array, and wash if necessary to remove unbound nucleic acid targets and analyse the results.

In one embodiment the readout from the analysis is fluorescence.

In one embodiment the readout from the analysis is colorimetric.

In one embodiment physical detection methods, such as changes in electrical impedance, nanowire technology or microfluidics may be used.

In one embodiment there is provided a method which further comprises the step of quantifying RNA from the subject sample.

If a quality control step is desired, software such as Genome Studio software may be employed.

Numeric value as employed herein is intended to refer to a number obtained for each relevant gene, from the analysis or readout of the gene expression, for example the fluorescence or colorimetric analysis. The numeric value obtained from the initial analysis may be manipulated, corrected and if the result of the processing is a still a number then it will be continue to be a numeric value.

By converting is meant processing of a negative numeric value to make it into a positive value or processing of a positive numeric value to make it into a negative value by simple conversion of a positive sign to a negative or vice versa.

Analysis of the subject-derived sample will for the genes analysed will give a range of numeric values some of which are positive (preceded by + and in mathematical terms considered greater than zero) and some of which are negative (preceded by − and in strict mathematical terms are considered to less than zero). The positive and negative in the context of gene expression analysis is a convenient mechanism for representing genes which are up-regulated and genes which are down regulated.

In the method of the present disclosure either all the numeric values of genes which are down-regulated and represented by a negative number are converted to the corresponding positive number (i.e. by simply changing the sign) for example −1 would be converted to 1 or all the positive numeric values for the up-regulated genes are converted to the corresponding negative number.

The present inventors have established that this step of rendering the numeric values for the gene expressions positive or alternatively all negative allows the summating of the values to obtain a single value that is indicative of the presence of disease or infection or the absence of the same.

This is a huge simplification of the processing of gene expression data and represents a practical step forward thereby rendering the method suitable for routine use in the clinic.

By discriminatory power is meant the ability to distinguish between a TB infected and a non-infected sample (subject) or between active TB infection and other infections (such as HIV) in particular those with similar symptoms or between a latent infection and an active infection.

The discriminatory power of the method according to the present disclosure may, for example, be increased by attaching greater weighting to genes which are more significant in the signature, even if they are expressed at low or lower absolute levels.

As employed herein, raw numeric value is intended to, for example refer to unprocessed fluorescent values from the gene chip, either absolute fluorescence or relative to a house keeping gene or genes.

Summating as employed herein is intended to refer to act or process of adding numerical values.

Composite expression score as employed herein means the sum (aggregate number) of all the individual numerical values generated for the relevant genes by the analysis, for example the sum of the fluorescence data for all the relevant up and down regulated genes. The score may or may not be normalised and/or scaled and/or weighted.

In one embodiment the composite expression score is normalised.

In one embodiment the composite expression score is scaled.

In one embodiment the composite expression score is weighted.

Weighted or statistically weighted as employed herein is intended to refer to the relevant value being adjusted to more appropriately reflect its contribution to the signature.

In one embodiment the method employs a simplified risk score as employed in the examples herein.

Simplified risk score is also known as disease risk score (DRS).

Control as employed herein is intended to refer to a positive (control) sample and/or a negative (control) sample which, for example is used to compare the subject sample to, and/or a numerical value or numerical range which has been defined to allow the subject sample to be designated as positive or negative for disease/infection by reference thereto.

Positive control sample as employed herein is a sample known to be positive for the pathogen or disease in relation to which the analysis is being performed, such as active TB.

Negative control sample as employed herein is intended to refer to a sample known to be negative for the pathogen or disease in relation to which the analysis is being performed.

In one embodiment the control is a sample, for example a positive control sample or a negative control sample, such as a negative control sample.

In one embodiment the control is a numerical value, such as a numerical range, for example a statistically determined range obtained from an adequate sample size defining the cut-offs for accurate distinction of disease cases from controls.

Conversion of multi-gene transcript disease signatures into a single number disease score

Once the RNA expression signature of the disease has been identified by variable selection, the transcripts are separated based on their up- or down-regulation relative to the comparator group. The two groups of transcripts are selected and collated separately.

### Summation of Up-Regulated and Down-Regulated RNA Transcripts

To identify the single disease risk score for any individual patient, the raw intensities, for example fluorescent intensities (either absolute or relative to housekeeping standards) of all the up-regulated RNA transcripts associated with the disease are summated. Similarly summation of all down-regulated transcripts for each individual is achieved by combining the raw values (for example fluorescence) for each transcript relative to the unchanged housekeeping gene standards. Since the transcripts have various levels of expression and respectively their fold changes differ as well, instead of summing the raw expression values, they can be scaled and normalised between 0,1. Alternatively they can be weighted to allow important genes to carry greater effect. Then, for every sample the expression values of the signature's transcripts are summated, separately for the up- and down-regulated transcripts.

The total disease score incorporating the summated fluorescence of up- and down-regulated genes is calculated by adding the summated score of the down-regulated transcripts (after conversion to a positive number) to the summated score of the up-regulated transcripts, to give a single number composite expression score. This score maximally distinguishes the cases and controls and reflects the contribution of the up- and down-regulated transcripts to this distinction.

### Comparison of the Disease Risk Score in Cases and Controls

The composite expression scores for patients and the comparator group may be compared, in order to derive the means and variance of the groups, from which statistical cut-offs are defined for accurate distinction of cases from controls. Using the disease subjects and comparator populations, sensitivities and specificities for the disease risk score may be calculated using, for example a Support Vector Machine and internal elastic net classification.

Disease risk score as employed herein is an indicator of the likelihood that patient has active TB when comparing their composite expression score to the comparator group's composite expression score.

Development of the Disease Risk Score into a Simple Clinical Test for Disease Severity or Disease Risk Prediction

The approach outlined above in which complex RNA expression signatures of disease or disease processes are converted into a single score which predicts disease risk can be used to develop simple, cheap and clinically applicable tests for disease diagnosis or risk prediction.

The procedure is as follows: For tests based on differential gene expression between cases and controls (or between different categories of cases such as severity), the up- and down-regulated transcripts identified as relevant may be printed onto a suitable solid surface such as microarray slide, bead, tube or well.

Up-regulated transcripts may be co-located separately from down-regulated transcripts either in separate wells or separate tubes. A panel of unchanged housekeeping genes may also be printed separately for normalisation of the results.

RNA recovered from individual patients using standard recovery and quantification methods (with or without amplification) is hybridised to the pools of up- and down-regulated transcripts and the unchanged housekeeping transcripts.

Control RNA is hybridised in parallel to the same pools of up- or down-regulated transcripts.

Total value, for example fluorescence for the subject sample and optionally the control sample is then read for up- and down-regulated transcripts and the results combined to give a composite expression score for patients and controls, which is/are then compared with a reference range of a suitable number of healthy controls or comparator subjects.

### Correcting the Detected Signal for the Relative Abundance of RNA Species in the Subject Sample

The details above explain how a complex signature of many transcripts can be reduced to the minimum set that is maximally able to distinguish between patients and other phenotypes. For example, within the up-regulated transcript set, there will be some transcripts that have a total level of expression many fold lower than that of others. However, these transcripts may be highly discriminatory despite their overall low level of expression. The weighting derived from the elastic net coefficient can be included in the test, in a number of different ways. Firstly, the number of copies of individual transcripts included in the assay can be varied. Secondly, in order to ensure that the signal from rare, important transcripts are not swamped by that from transcripts expressed at a higher level, one option would be to select probes for a test that are neither overly strongly nor too weakly expressed, so that the contribution of multiple probes is maximised. Alternatively, it may be possible to adjust the signal from low-abundance transcripts by a scaling factor.

Whilst this can be done at the analysis stage using current transcriptomic technology as each signal is measured separately, in a simple colorimetric test only the total colour change will be measured, and it would not therefore be possible to scale the signal from selected transcripts. This problem can be circumnavigated by reversing the chemistry usually associated with arrays. In conventional array chemistry, the probes are coupled to a solid surface, and the amount of biotin-labelled, patient-derived target that binds is measured. Instead, we propose coupling the biotin-labelled cRNA derived from the patient to an avidin-coated surface, and then adding DNA probes coupled to a chromogenic enzyme via an adaptor system. At the design and manufacturing stage, probes for low-abundance but important transcripts are coupled to greater numbers, or more potent forms of the chromogenic enzyme, allowing the signal for these transcripts to be ‘scaled-up’ within the final single-channel colorimetric readout. This approach would be used to normalise the relative input from each probe in the up-regulated, down-regulated and housekeeping channels of the kit, so that each probe makes an appropriately weighted contribution to the final reading, which may take account of its discriminatory power, suggested by the weights of variable selection methods.

The detection system for measuring multiple up or down regulated genes may also be adapted to use rTPCR to detect the transcripts comprising the diagnostic signature, with summation of the separate pooled values for up and down regulated transcripts, or physical detection methods such as changes in electrical impedance. In this approach, the transcripts in question are printed on nanowire surfaces or within microfluidic cartridges, and binding of the corresponding ligand for each transcript is detected by changes in impedance or other physical detection system

The present disclosure extends to a custom made chip comprising a minimal discriminatory gene set for diagnosis of active TB from other conditions, in particular those with similar symptoms, for example comprising at least 60-100% of the 27 genes listed in Table 3, and/or 60-100% of the 44 genes listed in Table 4, and/or 60-100% of the 53 genes listed in Table 5.

In one embodiment the gene chip is a fluorescent gene chip that is to say the readout is fluorescence.

Fluorescence as employed herein refers to the emission of light by a substance that has absorbed light or other electromagnetic radiation.

Thus in an alternate embodiment the gene chip is a colorimetric gene chip, for example colorimetric gene chip uses microarray technology wherein avidin is used to attach enzymes such as peroxidase or other chromogenic substrates to the biotin probe currently used to attach fluorescent markers to DNA. The present disclosure extends to a microarray chip adapted to read by colorimetric analysis and adapted for the analysis of active TB infection in a patient. The present disclosure also extends to use of a colorimetric chip to analyse a subject sample for active TB infection.

Colorimetric as employed herein refers to as assay wherein the output is in the human visible spectrum.

In an alternative embodiment, a gene set indicative of active TB may be detected by physical detection methods including nanowire technology, changes in electrical impedance, or microfluidics.

The readout for the assay can be converted from a fluorescent readout as used in current microarray technology into a simple colorimetric format or one using physical detection methods such as changes in impedance, which can be read with minimal equipment. For example, this is achieved by utilising the Biotin currently used to attach fluorescent markers to DNA. Biotin has high affinity for avidin which can be used to attach enzymes such as peroxidase or other chromogenic substrates. This process will allow the quantity of cRNA binding to the target transcripts to be quantified using a chromogenic process rather than fluorescence. Simplified assays providing yes/no indications of disease status can then be developed by comparison of the colour intensity of the up- and down-regulated pools of transcripts with control colour standards. Similar approaches can enable detection of multiple gene signatures using physical methods such as changes in electrical impedance.

This aspect of the invention is likely to be particularly advantageous for use in remote or under-resourced settings or for rapid diagnosis in “near patient” tests. For example, places in Africa because the equipment required to read the chip is likely to be simpler.

Multiplex assay as employed herein refers to a type of assay that simultaneously measures several analytes (often dozens or more) in a single run/cycle of the assay. It is distinguished from procedures that measure one analyte at a time.

In one embodiment there is provided a bespoke gene chip for use in the method, in particular as described herein.

In one embodiment there is provided use of a known gene chip for use in the method described herein in particular to identify one or more gene signatures described herein.

In one embodiment there is provided a method of treating latent TB after diagnosis employing the method disclosed herein.

In one embodiment there is provided a method of treating active TB after diagnosis employing the method disclosed herein.

Gene signature, gene set, disease signature, diagnostic signature and gene profile are used interchangeably throughout and should be interpreted to mean gene signature.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Method

### Study Sites and Patient Cohorts

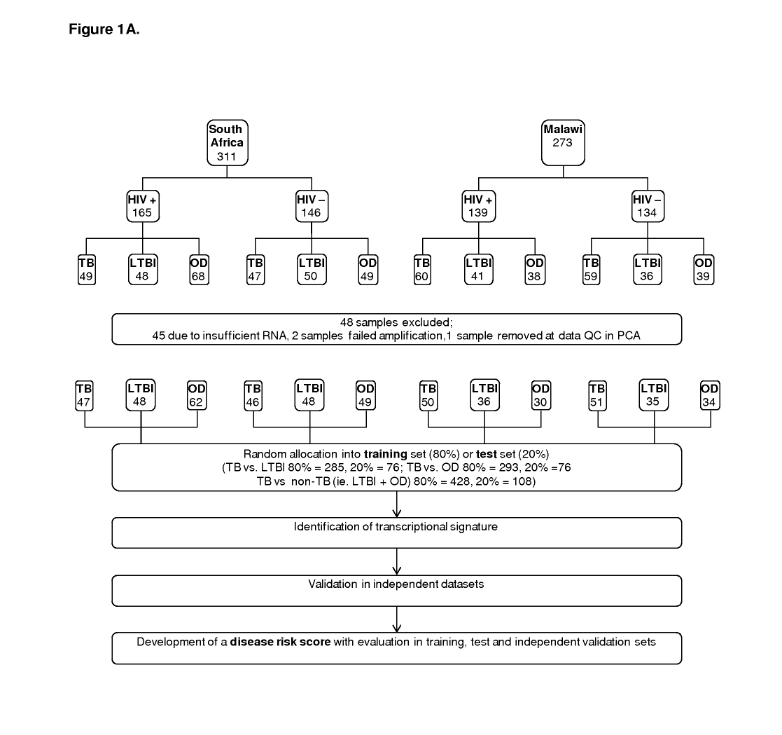

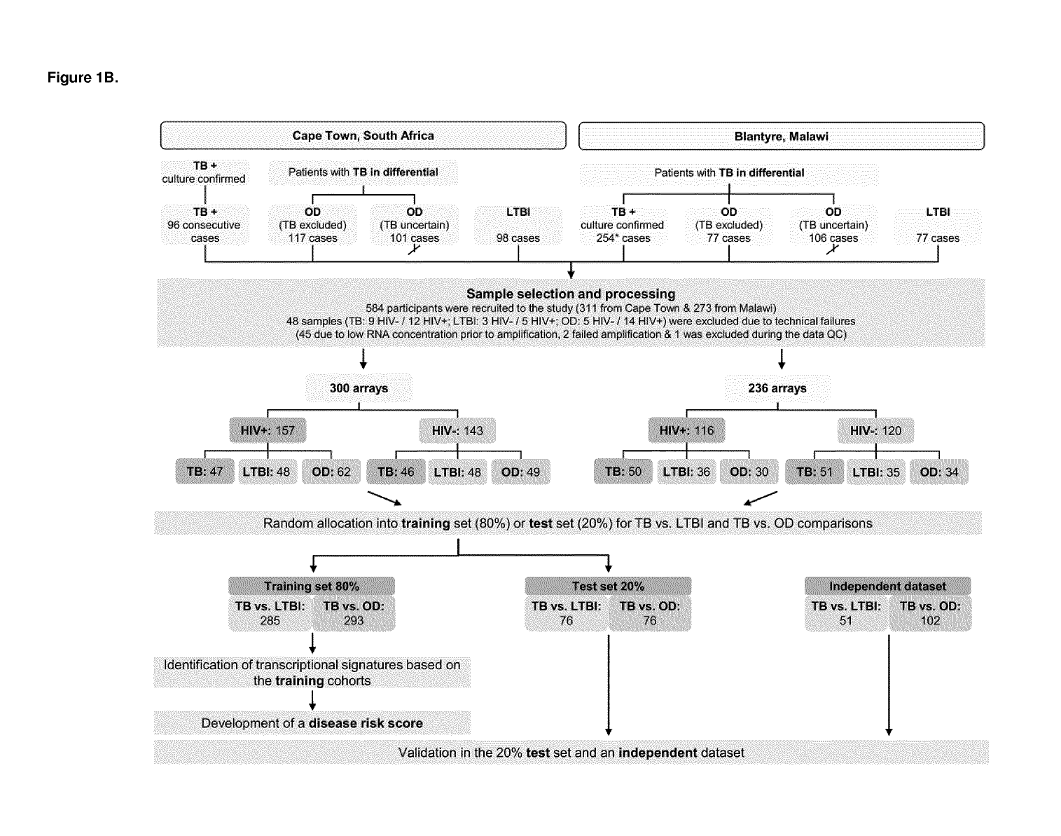

The overall plan of the study is shown in FIG. 1. In order to enable generalization of our findings to African countries with differing prevalence of malaria and other parasitic infections, as well as other environmental exposures that might affect transcriptional profiles, we chose highly contrasting study sites (one urban, one rural) in two African countries with differing co-endemic diseases (that is, where two or more diseases are endemic).

Cape Town, South Africa (SA):

SA has one of the highest TB incidence rates in Africa (981 per 100,000), as well as high rates of HIV infection (up to 41.8% prevalence in females aged 25-35). Patients undergoing investigation for suspected TB were recruited at GF Jooste Hospital Manenberg, Groote Schuur Hospital and at Khayelitsha site B, clinics serving the largely Xhosa population residing in the low income townships of Cape Town. Malaria is not endemic in these urban populations.

Karonga, Northern Malawi:

The incidence of new tuberculosis cases in Karonga district (180 per 100,000, Karonga Prevention Study unpublished data 2012) and the stable HIV prevalence (10-15% of females aged 25-29, Karonga Prevention Study unpublished data 2012) are lower in Karonga than Cape Town, and malaria and helminth infection are hyperendemic (that is, there is a high and continued incidence of disease). Patients were recruited at Karonga District hospital which serves a rural population living by the shores of Lake Malawi.

### Diagnostic Process

To ensure accurate assignment of patients to definite TB and OD groups, a rigorous diagnostic process was followed. All patients underwent chest radiographs and serological testing for HIV, along with cultures of blood, CSF and urine, and biopsies for histological examination including TB culture where clinically indicated. Two sputum samples obtained after induction or coughing were examined by standard microscopy for acid fast bacilli (AFB) and cultured for TB using standard methods (Crampin et al 2001). Patients were followed up 26 weeks post diagnosis to confirm that those with other diseases remained TB-free. Healthy LTBI controls were recruited by random community selection (Malawi) and from HIV screening clinics (SA) from the same catchment areas as patients with TB (FIG. 1). In vitro IGRA to substantiate LTBI was undertaken using an in-house whole blood assay (Hussain et al 2002; Franken et al 2000). Individuals were either assigned to one of the diagnostic groups or excluded once the results of investigations and follow-up were available. ‘Other Disease (OD)’ patients were recruited if they presented with symptoms that would mandate investigation for TB as a differential diagnosis. After intensive investigation, any case with an established alternative diagnosis to TB, no microbiological evidence of TB and an absence of TB symptoms at the time of follow-up or with an observed improvement of clinical symptoms on follow-up without TB treatment, was recruited as an OD case. If TB could not be reliably ruled out of the differential, the patient was excluded.

Following the diagnostic work-up, patients were assigned to groups using the following definitions (FIG. 1):

Definite TB case (TB): a participant with a clinical condition consistent with tuberculosis, and mycobacteria confirmed to be M.TB complex cultured from sputum or tissue samples. Confirmation of mycobacterial species was undertaken by Gen-Probe assay (Roche).

Latent TB infected case (LTBI): a participant who is clinically assessed as healthy and not suffering from a clinical syndrome in which tuberculosis is likely. The individuals will have a TST of 10 mm or more if HIV-uninfected, or 5 mm or more if HIV-infected and a positive IGRA and negative sputum culture. Sputum was only collected if the cough was productive, when at least two samples were collected. LTBI criteria were relaxed in the second year of the study to allow a positive TST and/or a positive IGRA to facilitate recruitment in Malawi. This change was made prior to any RNA expression measurements.

Other disease case (OD): A participant with a disease syndrome that on presentation includes tuberculosis in the differential diagnosis, but following clinical investigation and management, tuberculosis was excluded and a firm alternative diagnosis established.

Between January 2007 and June 2011, we recruited patients with suspected TB or other diseases (OD) in which the assessing clinician considered TB to be within the differential diagnosis. All patients underwent chest radiographs and serological testing for HIV, TST, cultures of blood, CSF and urine, and biopsies for histological examination (including TB culture where clinically indicated). Two sputum samples obtained after induction or coughing (Crampin et al 2001) were examined by standard microscopy for acid fast bacilli (AFB) and cultured for TB. Confirmation of mycobacterial species was undertaken by Gen-Probe assay (Roche). Patients were followed to confirm that those with OD remained TB-free for 26 weeks post diagnosis.

Healthy LTBI controls were recruited by random community selection (Malawi) and from HIV screening clinics (SA) from the same catchment areas as TB cases. In vitro IGRA to substantiate LTBI was undertaken using an in-house whole blood assay (Hussain et al 2002) (ESAT6 and CFP10 (Franken et al 2000) antigens supplied by THO). A rigorous diagnostic process and group definitions were implemented to ensure accurate assignment to TB, LTBI and OD groups (FIGS. 4A and 4B). Individuals were either assigned to one of 6 diagnostic groups or excluded once the results of investigations and follow-up were available (FIG. 1). Clinical and demographic features of recruited patients and the range of diagnoses in the OD group are shown in Table 1A and Table 1B.

### Ethical Approval and Consent

The study was approved by the Human Research Ethics Committee of the University of Cape Town, South Africa (HREC012/2007), the National Health Sciences Research Committee, Malawi NHSRC/447), and the Ethics Committee of the London School of Hygiene and Tropical Medicine (5212). Written information was provided by trained local health workers in local languages and all patients provided written consent.

### Oversight and Conduct of the Study

Patients were recruited to the study by local health care workers. Assignment of patients to clinical groups was made by consensus of experienced clinicians at each site (independent of those managing the patient clinically) after review of the investigation results. Testing for HIV status was conducted after appropriate counselling. Clinical data was anonymised and patient samples were identified only by study number. Microarrays were conducted by laboratory personnel blinded to assigned patient diagnostic groups. Statistical analysis was conducted only after the RNA expression data and clinical databases had been locked and deposited for independent verification.

### Peripheral Whole Blood RNA Expression by Microarray

Whole blood was collected at the time of recruitment (either before or within 24 hours of commencing TB treatment in suspected cases) in PAXgene® tubes, frozen within 3 hours of collection and later extracted using PAXgene® blood RNA extraction kits (Qiagen). RNA was shipped frozen to the Genome Institute of Singapore for analysis on HumanHT-12 v4 Expression BeadChips (Illumina).

Whole blood (2.5 ml) was collected into PAXgene™ blood RNA tubes (PreAnalytiX, Germany), incubated for 2 hours, frozen at −20° C. within 3 hours of collection, and then stored at −80° C. RNA was extracted using PAXgene™ blood RNA kits (PreAnalytiX, Germany) according to the manufacturer's instructions at one site (Cape Town) to minimize any sample handling bias. The integrity and yield of the total RNA was assessed using an Agilent 2100 Bioanalyser and a NanoDrop 1000 spectrophotometer respectively. Total RNA was then shipped to the Genome Institute of Singapore. After quantification and quality control, biotin-labelled cRNA was prepared using Illumina TotalPrep RNA Amplification kits (Applied Biosystems) from 500 ng RNA. Labelled cRNA was hybridized overnight to Human HT-12 V4 Expression BeadChip arrays (Illumina). After washing, blocking and staining, the arrays were scanned using an Illumina BeadArray Reader according to the manufacturer's instructions. Using Genome Studio software the microarray images were inspected for artefacts and QC parameters were assessed. No arrays were excluded at this stage.

### Statistical Analysis

Expression data were analysed using R′ Language and Environment for Statistical Computing (R) 2.12.1. To identify transcript signatures applicable across geographic locations and in patients with differing HIV status, we combined HIV-infected and -uninfected patient cohorts from SA and Malawi. The recruited subjects were randomly assigned to a “training” cohort (80% of the subjects) and a test cohort (20%) with no overlap. For additional validation we used the whole blood expression dataset of Berry et al. comparing TB with LTBI and other infections in an UK and an Africa cohort (accession GSE19491).

To detect transcripts that were differentially expressed between TB cases and comparator groups, a linear model was fitted and moderated t-statistics calculated for each transcript with correction for false discovery using Benjamini and Hochberg's method (1995). To identify the smallest number of transcripts distinguishing TB from the comparator groups, significantly differentially expressed (SDE) transcripts in the discovery cohort with a log 2 fold change (FC)>0.5 were subjected to variable selection using elastic net. These minimal transcript selected sets for TB vs. LTBI, TB vs. OD and TB vs. LTBI+OD were assessed in the test cohort and further evaluated using independent datasets (Berry et al 2010).

Mean raw intensity values for each probe were corrected for local background intensities and a robust spline normalisation (combining quantile normalisation and spline interpolation) was applied to each array. Expression values were transformed to a logarithmic scale (base 2), and for each probe. Differential expression between patient groups was identified by fitting a linear model to each transcript using LIMMA2. P-values were adjusted using the method of Benjamini and Hochberg. Transcripts with log FC >0.5 were taken forward to variable selection with elastic net. This threshold was chosen in order to ensure that differential expression for selected variables could be distinguished using the resolution of qtPCR. The a and X parameters of elastic net, which control the size of the selected model, were optimized via ten-fold cross-validation (CV). The weights assigned by elastic net to the trained model were used within a linear regression model to classify samples in the test set.

### A Simplified Method for Identifying Individual Patient's Risk of Active TB

Current whole genome array-based technologies are not well suited for use in resource poor settings as they are costly and require sophisticated technology as well as bioinformatics expertise. We therefore developed a method for translation of multiple transcript RNA signatures into a disease risk score, which could form the basis of a simple, low cost, diagnostic test requiring basic laboratory facilities and minimal bioinformatics analysis. For each individual, we calculated the disease risk score using the minimal transcript selected sets for TB vs. LTBI, TB vs. OD and TB vs. LTBI+OD. The score is derived by adding the total intensity at up-regulated transcripts, and subtracting the total intensity at all down-regulated transcripts. The sensitivity and specificity of this score in disease classification was evaluated on test and validation cohorts.

$$Threshold = \frac{( \frac{{μ}_{1}}{{σ}_{1}} + \frac{{μ}_{2}}{{σ}_{2}} )}{( \frac{1}{{σ}_{1}} + \frac{1}{{σ}_{2}} )}$$

Where μₙ is the mean of comparator group n, and σₙ is the standard deviation of comparator group n. The performance of the simplified risk score was then evaluated in our cohort as well as the independent datasets.

### Disease Risk Score

For each individual, we calculated the disease risk score using the minimal transcript selected sets for TB vs. LTBI, TB vs. OD and TB vs. LTBI+OD. The score is based on subtracting the summed intensities of the down-regulated transcripts from the summed intensities of the up-regulated transcripts. The risk score was calculated on normalised intensities. The disease risk score for individual i is:

$$Disease Risk {Score}^{i} = {\sum }_{k = 0}^{n} expr . {value}_{k}^{i} - {\sum }_{l = 0}^{m} expr . {value}_{l}^{i} \qquad ( 1 )$$

where: n the number of upregulated number of probes in the signature in disease of interest compared to comparator group(s).
m the number of downregulated number of probes in the signature in disease of interest compared to comparator group(s).

The threshold for the classification was calculated as the weighted average of risk score within each class, with weights given as inverse of the standard deviation of the score within each class (1/sd1 and 1/sd2 respectively). The threshold for the classification between group u and v is shown below:

$$threshold ( u , v ) = \frac{\frac{{μ}_{u}}{{σ}_{u}} + \frac{{μ}_{v}}{{σ}_{v}}}{\frac{1}{{σ}_{u}} + \frac{1}{{σ}_{v}}} \qquad ( 2 )$$

where: μ=average of the disease risk score in the group.
σ=standard deviation of the disease risk score in the group.

To calculate the indeterminate zone, we calculated the lower and upper threshold which were calculated as the weighted average with weights given by w/sd1, (1−w)/sd2 respectively for variable 0.5<w<=1. When w=0.5 its equivalent formula to main threshold. ROCs were generated using pROC₅.

Alternatively:

To calculate the indeterminate zone, we calculated the lower and upper threshold which were calculated as the weighted average with weights given

$$\frac{w}{{σ}_{u}} , \frac{2 - w}{{σ}_{v}}$$

respectively:

$${weighted}_{—} threshold ( u , v ) = \frac{w * \frac{{μ}_{u}}{{σ}_{u}} + ( 2 - w ) * \frac{{μ}_{v}}{{σ}_{v}}}{\frac{w}{{σ}_{u}} + \frac{2 - w}{{σ}_{v}}} , 0 \leq w \leq 2 \qquad ( 3 )$$

When w=1 the formula is equivalent to the main threshold formula.

### Evaluation of the Classification of the Disease Risk Score (DRS) and the Signatures

To evaluate the performance of the DRS as a classifier we used different measures (AUC, sensitivity, specificity, PPV, NPV, and likelihood ratios).

The calculation of the confidence intervals for the area under a receiver operating characteristic curve (AUC), the sensitivity and the specificity was based on a non-parametric stratified bootstrap resampling (each replicate contained the same number of cases and controls as the original sample) (Robin et al 2011), with 2000 bootstraps, as recommended by Carpenter et al. (2000). We also employed the exact binomial (Clopper et al 1934) to calculate the confidence intervals (Table 9).

We used the estimated sensitivity and specificity to calculate the positive and negative predictive values (PPV and NPV) using the following formulas:

$$PPV = \frac{sensitivity * prevalence}{sensitivity * prevalence + ( 1 - specificity ) * ( 1 - prevalence )}$$

$$NPV = \frac{specificity * ( 1 - prevalence )}{( 1 - sensitivity ) * prevalence + specificity * ( 1 - prevalence )}$$

and interpreting the prevalence as “the probability before the test is carried out that the subject has the disease” as suggested by D. Altman (1994). In this case, we assumed a clinical setting, such as the one used to recruit samples in Malawi, in which approximately 58% of patients with suspected TB had culture confirmed TB (254 TB confirmed cases/437 patients with suspected TB), as well as calculating more conservative values assuming a prevalence of 20% (as a more typical proportion would be 15%-25% in quality controlled laboratories in primary care settings in high-burden countries in sub-Saharan Africa). PPV and NPV can be interpreted as the probability that a sample with a positive test has active TB, and the probability that a sample with a negative test result does not have active TB respectively, and as such represent the diagnostic value of a test (Table S5). We also report positive and negative likelihood ratios along with their confidence intervals employing the method described in (Simel et al 1991) (Table 2A, 2B).

### Smaller Sets of Transcripts

Although the models suggested by elastic net were the smallest ones to provide us with the best classification, we wanted to further explore the performance of even smaller lists of transcripts. Instead of optimizing via ten-fold cross-validation (CV) both the α and λ parameters of elastic net which control the size of the selected model, we used α=1 which is the penalty for lasso that gives smaller models. Then, within the cross validation step of choosing λ, we forced the penalty to be such that the error would remain within one standard deviation of minimum error. This process resulted in 21 transcripts for the TB vs. LTBI comparison (12 overlapping with the 27 transcript signature) and 29 transcripts for the TB vs. OD comparison (14 overlapping with the 44 transcript signature). Smaller models have reduced sensitivity (6%-10% lower than the original models) while specificity remained the same (Table 11). When DRS was calculated sensitivity and specificity were 89% CI₉₅%[78-97] and 89% CI₉₅%[79-97] respectively for the TB vs. LTBI comparison. As for the TB vs. OD comparison, when DRS was calculated sensitivity and specificity were 83% CI₉₅%[69-93] and 88% CI₉₅%[76-97] respectively. Smaller models have mainly reduced sensitivity.

### Smear-Negatives

We have included 31 smear-negative patients with TB (with definite negative smear status) in the analysis of the adult cohort (7 TB HIV-uninfected and 24 TB HIV-infected). The TB/LTBI and the TB/OD DRSs were applied to these patients and as controls we used the LTBI and OD patients from the test set, while maintaining the same threshold. The performance of the TB/LTBI signature was comparable to the performance in the HIV-infected group and the performance of the TB/OD signature was almost the same as in the larger smear-negative and smear-positive group. Confidence intervals for the sensitivity and specificity of smear-negative patients with TB were calculated using both the bootstrapping and the exact binomial method (Table 12). These confidence intervals overlapped the corresponding CIs for the larger smear-positive and smear-negative group.

### Analysis of Validation Datasets

For validation of the performance of the disease risk score based on the TB vs. LTBI 27 transcript signature, TB vs. OD 44 transcript signature and TB vs. LTBI+OD 53 transcript signature, we used the whole blood expression dataset of Berry et al. generated using Illumina HT12 V3 Beadarrays comparing TB with LTBI and other infections in an UK and an Africa cohort (accession series GSE19491). For each testing dataset (UK GSE19444; SA GSE19442, OD GSE22098), both quantile and robust spline normalisation were applied separately to the arrays and the data was log transformed—however the results were the same regardless of normalisation method.

For the evaluation of the performance of our TB vs. LTBI 27 transcript signature, we used TB and LTBI patients in both of the normalized testing sets (UK TB n=21, LTBI n=21; SA TB n=20, LTBI n=31). The probe ILMN_{—}3247506 (FCGR1C) in the TB vs. LTBI signature was not on the HT12 V3 beadarray. For the evaluation of the performance of our 44 TB vs. OD transcript signature, we used TB patients from the normalized testing sets (UK testing TB n=21, SA TB n=20) and OD patients that did not include systemic lupus erythematosus as they were judged to be a rare disease in an African setting (n=82). The probes ILMN_{—}3287952 (LOC100133800), ILMN_{—}3215715 (LOC389386) and ILMN_{—}3308961 (MIR1974) in the TB vs. OD signature were not on the HT12 V3 beadchip.

For testing the performance of the reported 393 TB vs. LTBI signature and the 86 TB vs. OD signature on our African dataset, the disease risk score was calculated with these signatures as previously described, although 7 probes in the reported signatures were not present on the HT-12 V4 Beadchip (TB vs. LTBI 6 probes, TB vs. OD 1 probe).

In order to compare directly the differences of the performance of our signatures to the signatures presented in the Berry et al (2010), we calculated the differences of the means of the measures of classification (namely the AUC, the sensitivity and the specificity) on our test set along with their 95% confidence intervals, using the following mathematical formulas:

$$( a , b ) = {\hat{π}}_{1} - {\hat{π}}_{2} \pm {z}_{α \text{/} 2} \cdot s ( D ) s ( D ) = \sqrt{\frac{{\hat{π}}_{1} ( 1 - {\hat{π}}_{1} )}{{n}_{1}} + \frac{{\hat{π}}_{2} ( 1 - {\hat{π}}_{2} )}{{n}_{2}}}$$

### Biological Significance of the RNA Expression Data

The RNA signatures distinguishing TB from OD and LTBI were analysed through the use of IPA (Ingenuity® Systems, www.ingenuity.com), which identifies pathways and functions overrepresented in the datasets.

### Results

We recruited 311 adult patients to the South African cohort and 273 to the Malawi cohort (FIG. 1; Table 1A). After technical failures, 537 samples remained for analysis. The spectrum of infectious and malignant diseases in the OD cohorts reflected the range of conditions with similar clinical manifestations to TB at each site (Table 1B).

### Evidence for a TB Specific Signature Independent of Geographic Location and HIV Status

We performed quality control on the microarray data in order to examine the effect of disease state on the transcript expression and to check for assignment errors. Visual inspection revealed that the primary clustering was based on disease state (TB, LTBI, OD) rather than geographical location or HIV status (FIG. 5). There was substantial correlation of TB vs. LTBI differential expression across different geographic locations and HIV status which was also seen for TB vs. OD (FIGS. 6, 7). This indicates the presence of a robust underlying signature of TB, independent of HIV status or geographical location.

### Identification and Validation of Minimal Transcript-Sets

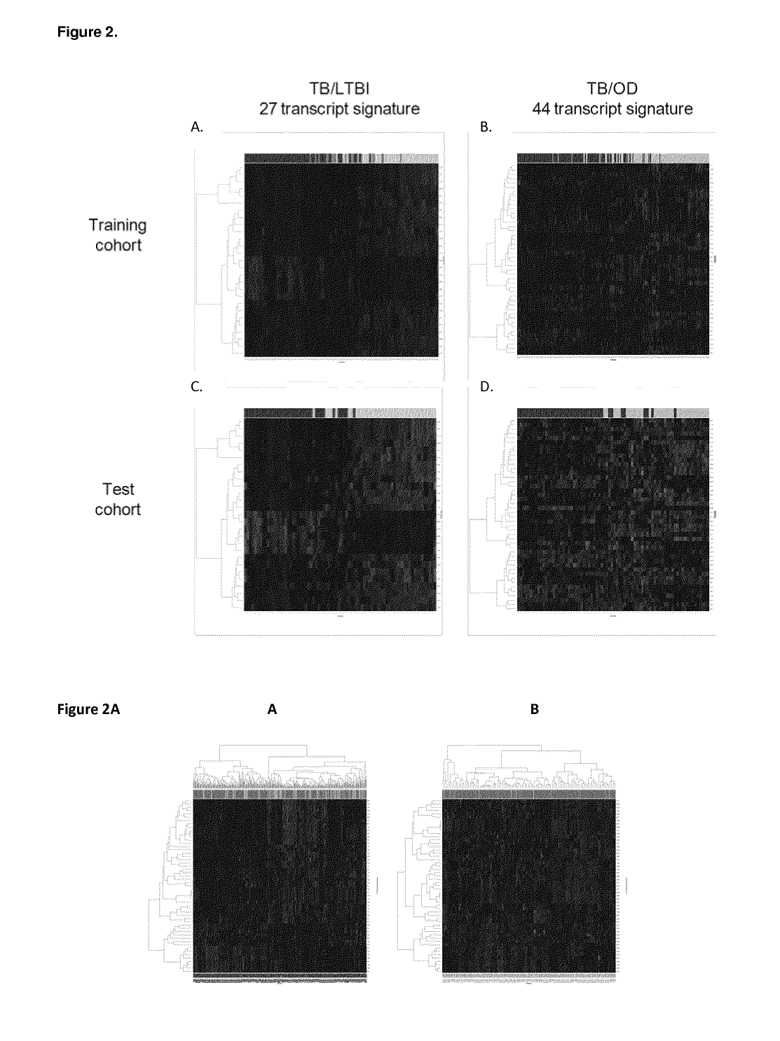

To find minimal transcript sets required to discriminate TB from other groups we applied the variable selection algorithm elastic net to the training cohort. A 27 transcript model was identified for discriminating TB from LTBI in the Malawi/SA training and test set (FIG. 2-A, 2-C, Table 3), whilst a 44 transcript model was identified for discriminating TB from OD (FIG. 2-B, 2-D, Table 4) and a 53 transcript model was identified for discriminating TB from LTBI+OD (FIG. 2A; Table 5). These signatures were also applied to data from the UK and the SA cohorts reported by Berry et al which, unlike our cohort, included only HIV-uninfected subjects (FIG. 8).

### Validation of the Minimal Gene Set on Test and an Independent Cohort

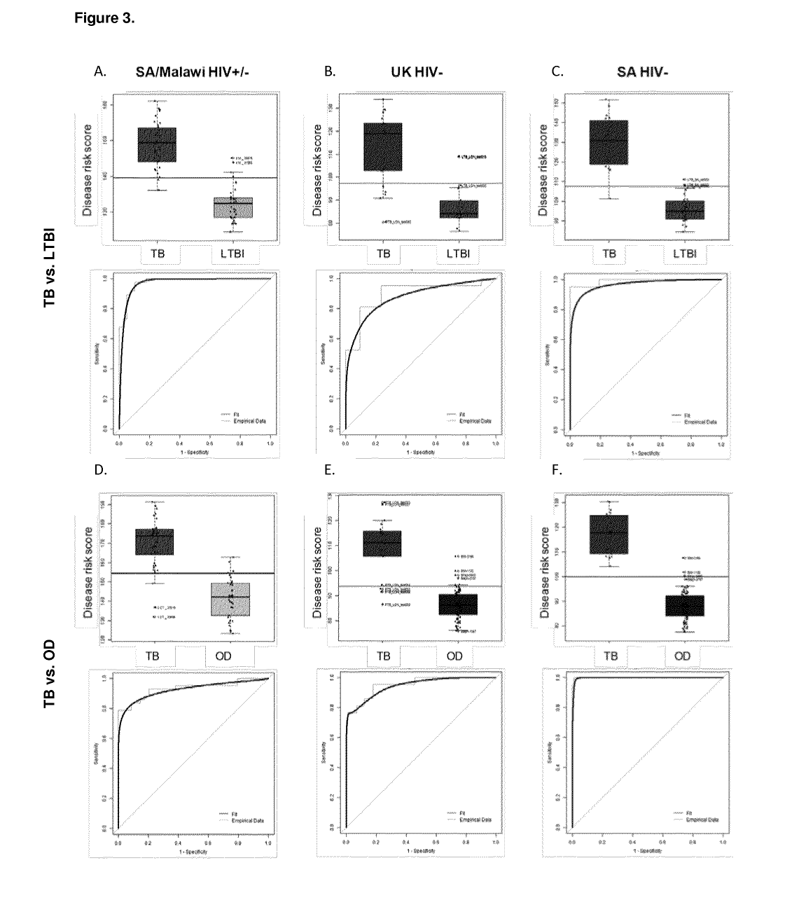

To evaluate the feasibility of using a simplified diagnostic test based on our transcript sets for TB diagnosis in low resource settings, we applied the disease risk score to our test cohort and to the UK and SA cohort data reported by Berry et al. In our combined HIV-infected and -uninfected test set, the 27 transcript disease risk score discriminated TB from LTBI with sensitivity and specificity of 95% and 90% respectively, whilst achieving perfect classification in the HIV-uninfected cohorts and slightly reduced accuracy in the HIV-infected cohorts (Table 2A, FIG. 3-A, FIG. 9). In the validation cohorts, the disease risk score performed better in the SA cohort than in the UK cohort (Table 2A, FIG. 3-B, 3-C). The 44 transcript disease risk score distinguished TB from OD with sensitivity and specificity of 93% and 88% respectively, with consistent accuracy in the HIV-uninfected and -infected cohorts (Table 2A, FIG. 3-D, FIG. 9). Classification was near perfect in the SA validation dataset while less accurate in the UK validation dataset (Table 2A, FIG. 3-E, 3-F). Similar values for sensitivity and specificity were obtained when the disease risk score was evaluated in the training dataset, demonstrating the robustness of our approach to overfitting (Table 6). Also, the disease risk score results are similar to those obtained using the regression model derived from the elastic net (Table 6).

In order to evaluate the classificatory power of the DRS, we compared its performance with the regression model derived from the elastic net based on the same signatures (Table 6). We found that our DRS had similar accuracy in distinguishing TB from LTBI and OD to the weighted regression model. In order to assess the predictive value of our DRS in a cohort of patients undergoing investigation for persistent symptoms such as cough, fever and weight loss i.e. where TB was included in the differential diagnosis, we used the prevalence of TB in our prospective Malawi cohort (58%; 254 confirmed TB cases of 437 patients with suspected TB) to calculate the positive and negative predictive value (PPV/NPV). The DRS for TB vs. OD had a PPV of 92% CI₉₅%[84-99] and a NPV of 90% CI₉₅%[80-100%] (Table 10). Using a 20% prevalence which may be more reflective of a general primary care setting in a high-burden African country, NPV for TB vs. OD is higher (98% CI₉₅%[96-100]), but PPV decreases (66% CI₉₅%[46-87]), emphasizing the value of DRS as a rule-out test, with those patients with positive DRS selected for further investigation (Table 10).

We also explored the effect of adjusting the threshold for the DRS in assigning individual patients to TB or LTBI/OD. By accepting a percentage of patients as ‘non-classifiable’, the majority of patients under investigation are accurately assigned. These ‘non-classifiable’ patients could then be selected for more detailed investigation (FIG. 11).

As it would be advantageous to have a single signature that distinguished TB from non-TB, we assessed the performance of a signature in distinguishing TB from both TB and LTBI. A 53 transcript signature was identified (Table 5) that distinguished TB from both LTBI and OD with sensitivity/specificity 91%/82%—a lower performance than TB/LTBI and TB/OD signatures alone. We also explored whether a smaller number of transcripts could be used to distinguish TB from LTBI and from OD which would aid in manufacturing of a test, resulting in a 21 and 29 probe signature for distinguishing TB from LTBI and OD respectively. The sensitivity of the smaller models was 6%-10% lower than the original models, while retaining the same specificity (Table 11).

In order to compare our minimal transcript signatures, derived from prospectively recruited African cohorts of HIV-infected and -uninfected patients with TB, OD and LTBI, with the previously reported signatures derived only from HIV-uninfected patients, and from OD that were not recruited during a prospective evaluation of patients in whom TB was included in the differential diagnosis, we compared the performance of our 27 probe TB/LTBI signature and our 44 probe TB/OD signature with the performance of the signatures of Berry et al. for discrimination of TB vs. LTBI (393 transcripts) and TB vs. OD (86 transcripts). While the 393 TB/LTBI signature achieved a sensitivity of 88% CI₉₅%[80-94] and a specificity of 84% CI₉₅%[76-92] on our TB HIV-uninfected cohorts, the performance on the HIV-infected group was 74% CI₉₅%[65-82] and 80% CI₉₅%[71-87] respectively (Table 2B, FIG. 10). Furthermore, the Berry et al. TB/OD 86 transcript signature had a lower performance on our cohorts (sensitivity 71% CI₉₅%[62-80], specificity 76% CI₉₅%[67-84] in HIV-uninfected; sensitivity 67% CI₉₅%[58-75], specificity 69% CI₉₅%[59-78] in HIV-infected; Table 2B, FIG. 10). Thus our minimal transcript signatures and the DRS method show better performance in distinguishing TB from LTBI and OD (especially in the HIV-infected cohorts) than the much larger number of probes identified by Berry et al. (Table 7).

We evaluated the performance of our signatures in the smear-negative sub-group of patients with TB, the majority of whom were HIV-infected (31 smear-negative TB patients with definite negative smear status; 7 TB HIV-uninfected and 24 TB HIV-infected). In the smear-negative patients the DRS showed a sensitivity for detecting TB of 68% CI₉₅%[52-84] when using the TB vs. LTBI signature and a sensitivity of 90% CI₉₅%[81-100] with the TB/OD signature, both of which are comparable to results obtained in the larger HIV-infected cohort of smear-positive and -negative patients. As we used the same LTBI and OD patients from the test set, the specificity was unchanged (90% CI₉₅%[80-97] for TB vs. LTBI and 88% CI₉₅%[74-97] for TB vs. OD, Table 12).

Finally, we also tested the signatures of Berry et al. for discrimination of TB vs. LTBI (393 transcripts) and TB vs. OD (86 transcripts) on our cohorts using the disease risk score. While the TB vs. LTBI signature gave good classification on our TB HIV-uninfected cohorts (sensitivity 88%; specificity 84%), the performance on the HIV-infected group was less good (sensitivity 74%; specificity 80%) (Table 2B, FIG. 10). The TB vs. OD signature showed poor discrimination on our cohorts (sensitivity 71%, specificity 76% in HIV-uninfected; sensitivity 67%, specificity 69% in HIV-infected) (Table 2B, FIG. 10). Thus the Berry signature is not applicable to a HIV infected cohort.

### Biological Significance of the TB Specific Probe Sets

Initial assignment (using IPA) of the 27 probe set distinguishing TB from LTBI, the 44 probe set distinguishing TB from OD, and the 53 probe set distinguishing TB from non TB, revealed that genes comprising each signature formed highly significant networks of genes that were involved in the inflammatory response, cell-to-cell signalling and interaction, as well as dendritic cell maturation (FIGS. 9-11).

### DISCUSSION

We have identified a host blood transcriptomic signature that distinguishes TB from a wide range of other conditions prevalent in HIV-infected and -uninfected Africans. We found that patients with TB can be distinguished from LTBI with only 27 transcripts, from OD with 44 transcripts and from LTBI and OD with 53 transcripts. Our finding appears robust as the results are reproducible in both HIV-infected and -uninfected cohorts, in different geographic locations, and in independent, publicly available datasets. The high sensitivity and specificity of our signatures in distinguishing TB from OD even in the HIV-infected patients that have differing levels of T cell depletion and a wide spectrum of opportunistic infections as well as HIV-related complications, suggest that the signatures are reliable markers of TB. The relatively small number of transcripts in our signatures suggests the potential to use RNA expression from a single peripheral blood sample as a clinical diagnostic tool (i.e. using a multiplex assay Joosten et al 2012, Eldering et al 2003).

Our signatures and the disease risk score accurately distinguish the majority of patients who have TB from those with OD and/or LTBI in whom TB is excluded.

Our study provides proof of principle that diagnosis of active TB in African countries affected by the HIV/TB epidemic is feasible using RNA expression on peripheral blood.

TABLE 1A
Clinical and diagnostic features of South Africa and Malawi cohorts with active
tuberculosis (TB), latent TB Infection (LTBI) or Other Diseases (OD).

| | Group | | | | | |
|---|---|---|---|---|---|---|
| | TB HIV+ | | TB HIV− | | LTBI HIV+ | |
| Location | SA | Malawi | SA | Malawi | SA | Malawi |
| Number | 49 | 60 | 47 | 59 | 48 | 41 |
| Age in years | 33.7 | 34.5 | 32.1 | 35.6 | 31.5 | 43.8 |
| Median (IQR) | (29.0-38.3) | (29.6-43.2) | (26.3-42.7) | (26.2-53.1) | (27.9-37.4) | (35.4-49.4) |
| Sex (male, %) | 40 | 52 | 70 | 58 | 27 | 22 |
| Duration of | 21 | 60 | 30 | 60 | NA | NA |
| symptoms/days | (0-33) | (14-210) | (21-30) | (30-240) | | |
| Median (IQR) | | | | | | |
| BMI (kg/m²) | 22.6 | 18.5 | 19.5 | 18.7 | 24.2 | 21.2 |
| Median (IQR) | (19.5-25.2) | (16.9-20.7) | (18.0-22.5) | (16.5-20.2) | (20.6-28.4) | (18.6-23.9) |
| CD4 count/mm³ | 174 | 128 | NA | NA | 326 | 312 |
| Median (IQR) | (64.7-293)¹ | (35-314) | | | (231-555) | (240-418) |
| Anti-retroviral | 4 | 14 | NA | NA | 1 | 0 |
| therapy | (8%) | (23.3%) | | | (2%) | (0%) |
| Tuberculin skin | 20 | ND | ND | ND | 16 | 17 |
| test induration | (15.5-22)³ | | | | (10-20) | (0-25) |
| (mm) Median (IQR) | | | | | | |
| IGRA positive (see | ND | ND | ND | ND | 48 | 22 |
| methods) | | | | | (100%) | (53.7%) |
| Malaria positive | NA | 2 | NA | 2 | NA | 1 |
| | | (3.3%) | | (3.4%) | | (2.4%) |
| | Group | | | | | |
| | LTBI HIV− | | OD HIV+ | | OD HIV− | |
| Location | SA | Malawi | SA | Malawi | SA | Malawi |
| Number | 50 | 36 | 68 | 38 | 49 | 39 |
| Age in years | 20.6 | 38.9 | 33.6 | 33.8 | 40.4 | 43.0 |
| Median (IQR) | (19.1-23.4) | (32.3-50.9) | (28.6-37.9) | (29.4-41.3) | (28.7-53.5) | (27.0-53.9) |
| Sex (male, %) | 42 | 53 | 38 | 34 | 45 | 28 |
| Duration of | NA | NA | 21 | 7 | 42 | 7 |
| symptoms/days | | | (6-90) | (3-90) | (7-130) | (2-365) |
| Median (IQR) | | | | | | |
| BMI (kg/m²) | 22.2 | 22.0 | 21.4 | 19.8 | 22.6 | 21.1 |
| Median (IQR) | (21.4-25.7) | (20.2-23.4) | (20.0-24.6) | (18.3-22.2) | (18.4-24.9) | (19.6-22.2) |
| CD4 count/mm³ | NA | NA | 197 | 198 | NA | NA |
| Median (IQR) | | | (92-357)² | (111-270) | | |
| Anti-retroviral | NA | NA | 26 | 16 | NA | NA |
| therapy | | | (38.2%) | (42.1%) | | |
| Tuberculin skin | 15 | 13 | ND | 0 | ND | 0 |
| test induration | (12-20) | (11-17) | | (0-0) | | (0-9) |
| (mm) Median (IQR) | | | | | | |
| IGRA positive (see | 50 | 13 | ND | ND | ND | ND |
| methods) | (100%) | (36.1%) | | | | |
| Malaria positive | NA | 0 | NA | 3 | NA | 2 |
| | | (0%) | | (7.9%) | | (5.1%) |

SA = South Africa, HIV− = HIV-uninfected, HIV+ = HIV-infected, IQR = inter quartile range, BMI = body mass index, TB = active TB, LTBI = latent TB infection, IGRA = Interferon gamma release assay, OD = other diseases (see below), ND = Not done, NA = Not applicable.
¹4 missing values.
²10 missing values.
³33 missing values, not routinely performed in the work up of TB+/HIV+ patients.

TABLE 1B
Major clinical diagnoses in ‘Other Diseases’ cohorts.

| | HIV | | HIV | | |
|---|---|---|---|---|---|
| | infected | | uninfected | | |
| | SA | Malawi | SA | Malawi | Total |
| Pneumonia/LRTI/PJP | 24 | 19 | 5 | 13 | 61 |
| Malignancy and other neoplasia other | 2 | 4 | 17 | 5 | 28 |
| than Kaposi's sarcoma^{†} | | | | | |
| Pelvic inflammatory disease/UTI | 4 | 1 | 15 | 5 | 25 |
| Bacterial, viral meningitis or | 4 | 4 | 0 | 6 | 14 |
| meningitis of uncertain origin | | | | | |
| Hepatobiliary disease | 6 | 0 | 7 | 0 | 13 |
| Febrile syndromes of uncertain origin | 1 | 3 | 1 | 6 | 11 |
| Kaposi's sarcoma | 9 | 1 | 0 | 0 | 10 |
| Cryptococcal meningitis | 6 | 4 | 0 | 0 | 10 |
| Non TB pleural effusion/Empyema | 5 | 0 | 2 | 0 | 7 |
| Gastroenteritis | 5 | 0 | 0 | 0 | 5 |
| Peritonitis | 0 | 1 | 0 | 3 | 4 |
| Other‡ | 0 | 1 | 2 | 1 | 4 |
| Gastric ulcer or gastritis | 2 | 0 | 0 | 0 | 2 |
| | 68 | 38 | 49 | 39 | 194 |

^{†}(14) Bronchial carcinoma, (4) Lymphoma, (1) Cervical carcinoma, (1) Ovarian carcinoma, (1) mesothelioma, (1) gastric carcinoma, (4) metastatic carcinoma of unknown origin, (1) benign salivary tumour, (1) Dermatological tumour
‡(1) HIV related lymphadenopathy, (1) Crohn's, (1) Orchitis, (1) Pyomyositis
LRTI = Lower respiratory tract infection, PJP = Pneumocystis jirovecii pneumonia, UTI = Urinary tract infection.

TABLE 2A
Classification achieved using the disease risk score.
The TB/LTBI 27 transcript signature and TB/OD 44 transcript
signature were applied to the South African/Malawi HIV-uninfected
(HIV−) and HIV-infected (HIV+) test cohort and the independent
validation dataset. Sensitivity and specificity calculated using
the weighted threshold for classification. The actual numbers of
patients that were DRS negative and positive are shown in Table S2.

| | South Africa/Malawi test cohort | | | Validation dataset | |
|---|---|---|---|---|---|
| | HIV+/− | HIV− | HIV+ | SA HIV− | UK HIV− |
| | (95% CI) | (95% CI) | (95% CI) | (95% CI) | (95% CI) |
| TB vs. latent TB | | | | | |
| infection (27 TB/LTBI | | | | | |
| transcript signature) | | | | | |
| Number of patients | n = 76 | n = 38 | n = 38 | n = 51 | |
| Area under the curve | 98% | 100% | 97% | 99% | 89% |
| | (95-100%) | (100-100%) | (95-100%) | (97-100%) | (88-96%) |
| Sensitivity | 95% | 100% | 94% | 95% | 76% |
| | (87-100%) | (100-100%) | (83-100%) | (85-100%) | (57-91%) |
| Specificity | 90% | 100% | 90% | 94% | 91% |
| | (80-97%) | (100-100%) | (75-100%) | (84-100%) | (76-100%) |
| Likelihood ratio | 9.23 | NA | 9.44 | 14.73 | |
| positive | (3.63-23.4) | | (2.52-5.34) | (3.84-56.47) | |
| Likelihood ratio | 0.06 | 0 | 0.06 | 0.05 | |
| negative | (0.02-0.23) | | (0.01-0.42) | (0.01-0.36) | |
| TB vs. Other Diseases | | | | | |
| (44 TB/OD transcript | | | | | |
| signature) | | | | | |
| Number of patients | n = 76 | n = 37 | n = 39 | n = 102 | |
| Area under the curve | 95% | 96% | 94% | 100%* | 95% |
| | (89-99%) | (89-100%) | (83-100%) | (100-100%) | (90-99%) |
| Sensitivity | 93% | 91% | 95% | 100% | 81% |
| | (83-100%) | (77-100%) | (85-100%) | (100-100%) | (62-95%) |
| Specificity | 88% | 93% | 84% | 96% | 92% |
| | (74-97%) | (80-100%) | (68-100%) | (93-100%) | (84-96%) |
| Likelihood ratio | 7.89 | 14.3 | 6.02 | 27.67 | |
| positive | (3.13-19.89) | (2.15-95.12) | (2.1-17.08) | (9.11-84.03) | |
| Likelihood ratio | 0.08 | 0.05 | 0.06 | 0 | |
| negative | (0.03-0.24) | (0.01-0.35) | (0.01-0.41) | | |

HIV− = HIV-uninfected,
HIV+ = HIV-infected,
NA = not applicable,
*99.94%

TABLE 2B
Application of published signatures to the South Africa and
Malawi cohorts.
Sensitivities, specificities and Area Under Curve based on tran-
script signatures of Berry et al. (2010) for TB vs. LTBI (393
probes), and TB vs. OD (86 probes) applied to the South African/
Malawi HIV-uninfected (HIV−) and HIV-infected (HIV+) cohorts.

| | South African/Malawi cohorts | | |
|---|---|---|---|
| | HIV−/+ | HIV− | HIV+ |
| | (95% CI) | (95% CI) | (95% CI) |
| TB vs. latent TB infection (27 | | | |
| TB/LTBI transcript signature) | | | |
| Number of patients | n = 361 | n = 180 | n = 181 |
| Area under the curve | 89% | 94% | 88% |
| | (86-92%) | (91-97%) | (82-92%) |
| Sensitivity | 82% | 88% | 74% |
| | (76-87%) | (80-94%) | (65-82%) |
| Specificity | 81% | 84% | 80% |
| | (75-87%) | (76-92%) | (71-87%) |
| TB vs. Other Diseases (44 | | | |
| TB/OD transcript signature) | | | |
| Number of patients | n = 369 | n = 180 | n = 189 |
| Area under the curve | 76% | 78% | 75% |
| | (70-80%) | (70-84%) | (68-82%) |
| Sensitivity | 68% | 71% | 67% |
| | (61-73%) | (62-80%) | (58-75%) |
| Specificity | 70% | 76% | 69% |
| | (62-76%) | (67-84%) | (59-78%) |

HIV− = HIV-uninfected, HIV+ = HIV-infected.

TABLE 3
27 gene signature

| | | | Direction of |
|---|---|---|---|
| Array ID | Gene Name | Probe ID | regulation* |
| 70730 | GAS6 | ILMN_1779558 | Up |
| 130181 | ANKRD22 | ILMN_1799848 | Up |
| 360132 | LHFPL2 | ILMN_1747744 | Up |
| 520086 | FCGR1A | ILMN_2176063 | Up |
| 1300139 | GNG7 | ILMN_1728107 | Down |
| 1340241 | C5 | ILMN_1746819 | Up |
| 1440341 | C1QC | ILMN_1785902 | Up |
| 1510026 | FLVCR2 | ILMN_2204876 | Up |
| 1780440 | CD79A | ILMN_1659227 | Down |
| 2630195 | VAMP5 | ILMN_1809467 | Up |
| 2650605 | C4ORF18 | ILMN_1672124 | Up |
| 2710709 | FCGR1B | ILMN_2261600 | Up |
| 2810373 | FAM20A | ILMN_1812091 | Up |
| 2970397 | ZNF296 | ILMN_1693242 | Down |
| 3520601 | MPO | ILMN_1705183 | Up |
| 3780047 | GBP6 | ILMN_1756953 | Up |
| 3890400 | CXCR5 | ILMN_2337928 | Down |
| 4280632 | GAS6 | ILMN_1784749 | Up |
| 5570039 | LOC728744 | ILMN_1654389 | Up |
| 5570398 | FCGR1C | ILMN_3247506 | Up |
| 5890470 | CCR6 | ILMN_1690907 | Down |
| 5910019 | C1QB | ILMN_1796409 | Up |
| 5910632 | SMARCD3 | ILMN_2309180 | Up |
| 6060468 | S100A8 | ILMN_1729801 | Up |
| 6450594 | CD79B | ILMN_1710017 | Down |
| 6560156 | DUSP3 | ILMN_1797522 | Up |
| 6620209 | FCGR1B | ILMN_2391051 | Up |

*in TB patients in relation to patients with latent TB infection.

TABLE 4
44 gene signature

| | | | Direction of |
|---|---|---|---|
| Array ID | Gene Name | Probe ID | regulation* |
| 130086 | CYB561 | ILMN_1771179 | Up |
| 150224 | LOC196752 | ILMN_1803743 | Up |
| 270039 | HM13 | ILMN_1766269 | Up |
| 360132 | LHFPL2 | ILMN_1747744 | Up |
| 380541 | PPPDE2 | ILMN_1737580 | Up |
| 450132 | RBM12B | ILMN_1805778 | Up |
| 450379 | PRDM1 | ILMN_2294784 | Up |
| 540041 | CASC1 | ILMN_1708983 | Up |
| 840446 | CYB561 | ILMN_2378376 | Up |
| 1030433 | CALML4 | ILMN_1815707 | Up |
| 1050360 | HLA-DPB1 | ILMN_1749070 | Up |
| 1070477 | ALDH1A1 | ILMN_2096372 | Up |
| 1110592 | EBF1 | ILMN_1778681 | Down |
| 1170332 | AAK1 | ILMN_1688755 | Up |
| 1580437 | PGA5 | ILMN_1717572 | Down |
| 1690184 | RNF19A | ILMN_1812327 | Up |
| 2000682 | HS.131087 | ILMN_1916292 | Down |
| 2030309 | SERPING1 | ILMN_1670305 | Up |
| 2260349 | MIR1974 | ILMN_3308961 | Up |
| 2340241 | IMPA2 | ILMN_2094061 | Down |
| 2350114 | GJA9 | ILMN_1710161 | Up |
| 2850315 | ORM1 | ILMN_1696584 | Down |
| 3120475 | MAP7 | ILMN_2216815 | Down |
| 3130600 | BTN3A1 | ILMN_1802708 | Up |
| 3310504 | PDK4 | ILMN_1684982 | Down |
| 3360553 | RP5-1022P6.2 | ILMN_1701111 | Down |
| 3780047 | GBP6 | ILMN_1756953 | Up |
| 3840053 | UGP2 | ILMN_1671969 | Up |
| 4070524 | CERKL | ILMN_1801091 | Up |
| 4290619 | CREB5 | ILMN_1728677 | Up |
| 4560047 | CD74 | ILMN_1761464 | Up |
| 4570164 | LOC389386 | ILMN_3215715 | Up |
| 4640768 | VPREB3 | ILMN_1700147 | Down |
| 4670458 | SEPT4 | ILMN_1776157 | Up |
| 5260161 | HS.162734 | ILMN_1893697 | Down |
| 5270753 | ARG1 | ILMN_1812281 | Down |
| 5290100 | MAK | ILMN_1803984 | Down |
| 5820491 | MAP7 | ILMN_1712719 | Down |
| 6380681 | C19ORF12 | ILMN_1664920 | Up |
| 6510754 | ALDH1A1 | ILMN_1709348 | Up |
| 6560156 | DUSP3 | ILMN_1797522 | Up |
| 6760056 | LOC100133800 | ILMN_3287952 | Up |
| 6760471 | TMCC1 | ILMN_1677963 | Down |
| 7210110 | HM13 | ILMN_2236655 | Up |

*in TB patients in relation to patients with other diseases.

TABLE 5
53 gene signature

| | | | Direction of |
|---|---|---|---|
| Array ID | Gene Name | Probe ID | regulation* |
| 70730 | GAS6 | ILMN_1779558 | Up |
| 130086 | CYB561 | ILMN_1771179 | Up |
| 130181 | ANKRD22 | ILMN_1799848 | Up |
| 360132 | LHFPL2 | ILMN_1747744 | Up |
| 380541 | PPPDE2 | ILMN_1737580 | Up |
| 520086 | FCGR1A | ILMN_2176063 | Up |
| 540041 | CASC1 | ILMN_1708983 | Up |
| 840446 | CYB561 | ILMN_2378376 | Up |
| 870408 | IL15 | ILMN_1724181 | Up |
| 1030433 | CALML4 | ILMN_1815707 | Up |
| 1070477 | ALDH1A1 | ILMN_2096372 | Up |
| 1090497 | CREG1 | ILMN_1680624 | Up |
| 1110592 | EBF1 | ILMN_1778681 | Down |
| 1300139 | GNG7 | ILMN_1728107 | Down |
| 1510364 | GBP5 | ILMN_2114568 | Up |
| 1580437 | PGA5 | ILMN_1717572 | Down |
| 1660021 | RNU4ATAC | ILMN_3240594 | Up |
| 1940274 | IFI27L2 | ILMN_1740319 | Up |
| 2000682 | ILMN_1916292 | ILMN_1916292 | Down |
| 2340682 | UHMK1 | ILMN_2096012 | Up |
| 2680136 | SIGLEC11 | ILMN_1674593 | Up |
| 2970747 | DEFA3 | ILMN_2165289 | Up |
| 3130600 | BTN3A1 | ILMN_1802708 | Up |
| 3190113 | VPS13B | ILMN_2268409 | Up |
| 3420259 | MIR21 | ILMN_3310840 | Up |
| 3780047 | GBP6 | ILMN_1756953 | Up |
| 3840053 | UGP2 | ILMN_1671969 | Up |
| 3840753 | HEY1 | ILMN_1788203 | Down |
| 3890400 | CXCR5 | ILMN_2337928 | Down |
| 4290619 | CREB5 | ILMN_1728677 | Up |
| 4540239 | DEFA1 | ILMN_2193213 | Up |
| 4560047 | CD74 | ILMN_1761464 | Up |
| 4570164 | LOC389386 | ILMN_3215715 | Up |
| 4640768 | VPREB3 | ILMN_1700147 | Down |
| 4670113 | LOC90925 | ILMN_1794927 | Down |
| 4670458 | SEPT4 | ILMN_1776157 | Up |
| 4860128 | DEFA1B | ILMN_1725661 | Up |
| 5260161 | ILMN_1893697 | ILMN_1893697 | Down |
| 5570398 | FCGR1C | ILMN_3247506 | Up |
| 5720180 | FZD2 | ILMN_1653711 | Up |
| 5820491 | MAP7 | ILMN_1712719 | Down |
| 6330471 | BLK | ILMN_1668277 | Down |
| 6380040 | COL9A2 | ILMN_1685122 | Down |
| 6380338 | POLB | ILMN_1767894 | Up |
| 6400414 | LOC650546 | ILMN_1814812 | Up |
| 6510754 | ALDH1A1 | ILMN_1709348 | Up |
| 6560156 | DUSP3 | ILMN_1797522 | Up |
| 6590646 | FAM26F | ILMN_2066849 | Up |
| 6620161 | SPIB | ILMN_2143314 | Down |
| 6620209 | FCGR1B | ILMN_2391051 | Up |
| 6760471 | TMCC1 | ILMN_1677963 | Down |
| 6760593 | OSBPL10 | ILMN_1669497 | Down |
| 7150170 | DEFA1B | ILMN_2102721 | Up |

*in TB patients in relation to patients with latent TB infection and other diseases.

TABLE 6

| | Elastic net | | | | | |
|---|---|---|---|---|---|---|
| | HIV+/− | | HIV− | | HIV+ | |
| | Training | Test | Training | Test | Training | Test |
| | (95% CI) | (95% CI) | (95% CI) | (95% CI) | (95% CI) | (95% CI) |
| TB vs. LTBI (27 TB/LTBI | | | | | | |
| transcript signature) | | | | | | |
| Area under the curve | 97% | 97% | 99% | 100% | 95% | 96% |
| | (95-98%) | (94-99%) | (98-100%) | (100-100%) | (91-98%) | (91-100%) |
| Sensitivity | 87% | 89% | 84% | 84% | 89% | 94% |
| | (81-92%) | (78-97%) | (75-92%) | (68-100%) | (81-96%) | (83-100%) |
| Specificity | 91% | 90% | 99% | 100% | 84% | 80% |
| | (86-96%) | (80-97%) | (96-100%) | (100-100%) | (75-91%) | (60-95%) |
| TB vs. Other Diseases (44 | | | | | | |
| TB/OD transcript signature) | | | | | | |
| Area under the curve | 97% | 94% | 97% | 95% | 97% | 94% |
| | (95-98%) | (88-99%) | (94-100%) | (88-100%) | (94-99%) | (84-100%) |
| Sensitivity | 93% | 83% | 95% | 82% | 92% | 85% |
| | (90-97%) | (71-93%) | (89-99%) | (64-96%) | (86-97%) | (70-100%) |
| Specificity | 89% | 97% | 90% | 100% | 88% | 95% |
| | (83-94%) | (91-100%) | (82-96%) | (100-100%) | (80-95%) | (84-100%) |

| | Disease risk score (DRS) | | | | | |
|---|---|---|---|---|---|---|
| | HIV+/− | | HIV− | | HIV+ | |
| | Training | Test | Training | Test | Training | Test |
| | (95% CI) | (95% CI) | (95% CI) | (95% CI) | (95% CI) | (95% CI) |
| TB vs. LTBI (27 TB/LTBI | | | | | | |
| transcript signature) | | | | | | |
| Area under the curve | 95% | 98% | 98% | 100% | 92% | 97% |
| | (93-97%) | (95-100%) | (97-100%) | (100-100%) | (88-96%) | (95-100%) |
| Sensitivity | 87% | 95% | 91% | 100% | 81% | 94% |
| | (81-92%) | (87-100%) | (85-97%) | (100-100%) | (72-90%) | (83-100%) |
| Specificity | 87% | 90% | 89% | 100% | 86% | 90% |
| | (81-92%) | (80-97%) | (81-95%) | (100-100%) | (77-94%) | (75-100%) |
| TB vs. Other Diseases (44 | | | | | | |
| TB/OD transcript signature) | | | | | | |
| Area under the curve | 96% | 95% | 97% | 96% | 95% | 94% |
| | (94-98%) | (89-99%) | (94-99%) | (89-100%) | (92-98%) | (83-100%) |
| Sensitivity | 88% | 93% | 89% | 91% | 86% | 95% |
| | (82-93%) | (83-100%) | (83-96%) | (77-100%) | (78-92%) | (85-100%) |
| Specificity | 87% | 88% | 88% | 93% | 86% | 84% |
| | (82-92%) | (74-97%) | (79-96%) | (80-100%) | (78-95%) | (68-100%) |

HIV− = HIV-uninfected, HIV+ = HIV-infected.

TABLE 6A
Classification achieved using elastic net derived linear classifier
with the 53 transcript-set identified for TB vs. non-TB (i.e.
LTBI and OD) when applied to the HIV-uninfected (HIV) and
HIV-infected (HIV+) training and test cohorts.

| | All (HIV+/−) | | HIV− | | HIV+ | |
|---|---|---|---|---|---|---|
| TB vs non-TB | Training | Test | Training | Test | Training | Test |
| Sensitivity | 81% | 91% | 87% | 100% | 75% | 84% |
| Specificity | 88% | 82% | 89% | 81% | 86% | 84% |

HIV+ = HIV-infected, HIV− = HIV-uninfected.

TABLE 7
Performance of the TB/LTBI 27 and TB/OD 44 transcript signatures and the transcript signatures of Berry et al. (2010) when applied to our
test cohort. Comparison of the statistical measures of performance of disease classification using our TB/LTBI 27 and TB/OD 44 transcript
signatures with the classification using the 393 (−6 transcript) and 86 (−1 transcript) transcript signatures from Berry et al. (2010).

| | South Africa/Malawi test cohort | | | | | | | | |
|---|---|---|---|---|---|---|---|---|---|
| | HIV+/− (95% CI) | | | HIV− (95% CI) | | | HIV+ (95% CI) | | |
| | Our | Berry et al. | | Our | Berry et al. | | Our | Berry et al. | |
| | signatures | signatures | Difference* | signatures | signatures | Difference* | signatures | signatures | Difference* |
| TB vs. LTBI | | | | | | | | | |
| Area under the curve | 98% | 88% | +10% | 100% | 91% | +9% | 97% | 89% | +9% |
| | (95-100%) | (85-97%) | (2-18%) | (100-100%) | (88-100%) | (0-18%) | (92-100%) | (83-98%) | (−3-20%) |
| Sensitivity | 95% | 84% | +11% | 100% | 90% | +11% | 94% | 78% | +17% |
| | (87-100%) | (73-95%) | (1-21%) | (100-100%) | (74-100%) | (1-20%) | (83-100%) | (61-94%) | (2-32%) |
| Specificity | 90% | 87% | +3% | 100% | 79% | +21% | 90% | 85% | +5% |
| | (80-97%) | (77-97%) | (−8-13%) | (100-100%) | (58-95%) | (8-34%) | (75-100%) | (65-100%) | (−10-20%) |
| TB vs. Other Diseases | | | | | | | | | |
| Area under the curve | 95% | 73% | +22% | 96% | 76% | +20% | 94% | 72% | +21% |
| | (89-99%) | (63-86%) | (10-33%) | (89-100%) | (62-91%) | (5-35%) | (82-100%) | (57-89%) | (5-37%) |
| Sensitivity | 93% | 74% | +19% | 91% | 77% | +14% | 95% | 70% | +25% |
| | (83-100%) | (60-86%) | (8-31%) | (77-100%) | (59-96%) | (−3-30%) | (85-100%) | (50-90%) | (9-41%) |
| Specificity | 88% | 74% | +15% | 93% | 67% | +27% | 84% | 74% | +11% |
| | (74-97%) | (59-88%) | (2-27%) | (80-100%) | (40-87%) | (9-44%) | (68-100%) | (53-90%) | (−7-28%) |

The marked improvement shown for HIV+ individuals in both TB vs. LTBI and TB vs. OD comparisons suggests that transcript signatures must be derived from both HIV-infected and -uninfected individuals in order to have a diagnostic value in these populations. The performance of our signatures in TB vs. OD comparison highlights the need for real world “other disease” controls when deriving biomarkers from clinical cohorts.
*Calculations of the differences were performed before rounding for reporting purposes on the paper.

TABLE 8
Number of patients per group and calls of DRS classification per group. Values of
sensitivity, specificity and their confidence intervals are presented in Table 2A.

| | | | | Validation |
|---|---|---|---|---|
| | South Africa/Malawi test cohort | | | dataset |
| | HIV+/− | HIV− | HIV+ | HIV− |
| | (95% CI) | (95% CI) | (95% CI) | (95% CI) |
| TB vs. latent TB infection (27 | | | | |
| TB/LTBI transcript signature) | | | | |
| Number of patients | n_{ALL} = 76; | n_{ALL} = 38; | n_{ALL} = 38; | n_{ALL} = 51; |
| | n_{TB} = 37; | n_{TB} = 19; | n_{TB} = 18; | n_{TB} = 20; |
| | n_{LTBI} = 39 | n_{LTBI} = 19 | n_{LTBI} = 20 | n_{LTBI} = 31 |
| Positive calls by DRS/ | [35/37] | [19/19] | [17/18] | [19/20] |
| Positive by gold standard | | | | |
| Negative calls by DRS/ | [35/39] | [19/19] | [18/20] | [29/31] |
| Negative by gold standard | | | | |
| TB vs. Other Diseases (44 | | | | |
| TB/OD transcript signature) | | | | |
| Number of patients | n_{ALL} = 76; | n_{ALL} = 37; | n_{ALL} = 39; | n_{ALL} = 102; |
| | n_{TB} = 42; | n_{TB} = 22; | n_{TB} = 20; | n_{TB} = 20; |
| | n_{OD} = 34 | n_{OD} = 15 | n_{OD} = 19 | n_{OD} = 83 |
| Positive calls by DRS/ | [39/42] | [20/22] | [19/20] | [20/20] |
| Positive by gold standard | | | | |
| Negative calls by DRS/ | [30/34] | [14/15] | [16/19] | [80/83] |
| Negative by gold standard | | | | |

TABLE 9
Classification achieved using the disease risk score applied
to the South African/Malawi HIV-uninfected (HIV−) and
HIV-infected (HIV+) test cohort with confidence intervals
calculated using the exact binomial method.

| | | | Validation |
|---|---|---|---|
| South Africa/Malawi test cohort | | | dataset |
| HIV+/− | HIV− | HIV+ | HIV− |
| (95% CI) | (95% CI) | (95% CI) | (95% CI) |

TB vs. latent TB infection (27 TB/LTBI transcript signature)

| | | | | |
|---|---|---|---|---|
| Sensitivity (mean) | 95% | 100% | 94% | 95% |
| | (82-99%) | (82-100%) | (73-100%) | (75-100%) |
| Specificity (mean) | 90% | 100% | 90% | 94% |
| | (76-96%) | (82-100%) | (68-99%) | (79-99%) |

TB vs. Other Diseases (44 TB/OD transcript signature)

| | | | | |
|---|---|---|---|---|
| Sensitivity (mean) | 93% | 91% | 95% | 100% |
| | (81-99%) | (71-99%) | (75-100%) | (83-100%) |
| Specificity (mean) | 88% | 93% | 84% | 96% |
| | (73-97%) | (68-100%) | (60-97%) | (90-99%) |

TABLE 10
Positive and Negative predictive values for the classification achieved using the disease risk score
applied to the South African/Malawi HIV-uninfected (HIV−) and HIV-infected (HIV+) test cohort.

| | South Africa/Malawi test cohort | | | | | | Validation dataset | |
|---|---|---|---|---|---|---|---|---|
| | HIV+/− | | HIV− | | HIV+ | | HIV− | |
| | PPV | NPV | PPV | NPV | PPV | NPV | PPV | NPV |
| | (95% CI) | (95% CI) | (95% CI) | (95% CI) | (95% CI) | (95% CI) | (95% CI) | (95% CI) |
| TB vs. LTBI (27 TB/LTBI | | | | | | | | |
| transcript signature) | | | | | | | | |
| 20% prevalence | 70% | 99% | 100% | 100% | 70% | 98% | 79% | 99% |
| | (50-89%) | (97-100%) | (100-100%) | (100-100%) | (42-99%) | (96-100%) | (56-100%) | (96-100%) |
| 58% prevalence | 93% | 92% | 100% | 100% | 93% | 92% | 95% | 93% |
| | (86-99%) | (83-100%) | (100-100%) | (100-100%) | (84-100%) | (78-100%) | (89-100%) | (81-100%) |
| TB vs. Other Diseases (44 | | | | | | | | |
| TB/OD transcript signature) | | | | | | | | |
| 20% prevalence | 66% | 98% | 77% | 98% | 60% | 99% | 87% | 100% |
| | (46-87%) | (96-100%) | (44-100%) | (94-100%) | (35-85%) | (96-100%) | (75-100%) | (100-100%) |
| 58% prevalence | 92% | 90% | 95% | 88% | 89% | 92% | 97% | 100% |
| | (84-99%) | (80-100%) | (86-100%) | (74-100%) | (79-99%) | (79-100%) | (95-100%) | (100-100%) |

TABLE 11
Performance of the smaller signatures when applied
to the South Africa/Malawi test set.

South Africa/Malawi test cohort

| | |
|---|---|
| Sensitivity | Specificity |

TB vs. latent TB infection

| | | |
|---|---|---|
| 27 transcript signature | 95% | 90% |
| | (87-100%) | (80-97%) |
| 21 transcript signature | 89% | 89% |
| | (78-97%) | (79-97%) |

TB vs. other diseases

| | | |
|---|---|---|
| 44 transcript signature | 93% | 88% |
| | (83-100%) | (74-97%) |
| 29 transcript signature | 83% | 88% |
| | (69-93%) | (75-97%) |

TABLE 12
Classification achieved using the disease risk score applied to
the South African/Malawi smear-negative patients with TB and the
controls from the test cohort with confidence intervals calculated
using the bootstrapping and the exact binomial method.

South Africa/Malawi smear negative TB
and controls from the test cohort

| | |
|---|---|
| Sensitivity | Specificity |

TB vs. latent TB infection
n_{ALL} = 70; n_{TB smear neg} = 31; n_{LTBI} = 39

| | | |
|---|---|---|
| Calls by DRS | [21/31] | [35/39] |
| Bootstrapping | 68% | 90% |
| | (52-84) | (80-97) |
| Exact Binomial | 68% | 90% |
| | (49-83) | (76-97) |

TB vs. other diseases
n_{ALL} = 65; n_{TB smear neg} = 31; n_{OD} = 34

| | | |
|---|---|---|
| Calls by DRS | [28/31] | [30/34] |
| Bootstrapping | 90% | 88% |
| | (81-100) | (74-97) |
| Exact Binomial | 90% | 88% |
| | (74-98) | (73-97) |

### REFERENCES

WHO report 2011 Global Tuberculosis Control 2011. (http://www.who.int/tb/publications/global_report/en/)
Schultz 2010 Integrative Genomic Profiling of Human Prostate Cancer Cancer Cell Vol 18, Issue 1, 11-22
Metcalfe et al 2010 (“Interferon-γ release assays for active pulmonary tuberculosis diagnosis in adults in low- and middle-income countries: systematic review and meta-analysis” The Journal of infectious diseases 204 Suppl 4).
Berry M P, Graham C M, McNab F W, et al. An interferon-inducible neutrophil-driven blood transcriptional signature in human tuberculosis. Nature 2010; 466:973-7.
Denoeud F, Aury J M, Da Silva C, et al, F; Artiguenave (2008). “Annotating genomes with massive-scale RNA sequencing”. Genome Biol. 9 (12): R175.
Velculescu V E, Zhang L, Vogelstein B, Kinzler K W. (1995) “Serial analysis of gene expression”. Science 270 (5235): 484-7.
Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003 April; 4(2):249-64.
Tusher, Virginia Goss; Tibshirani, Robert; Chu, Gilbert (2001). “Significance analysis of microarrays applied to the ionizing radiation response”. Proceedings of the National Academy of Sciences of the United States of America 98 (18): 5116-5121.
Zou, H., and Hastie, T. 2005. Regularization and variable selection via the elastic net. J Roy Stat Soc Ser B 67:301-320. The relevant algorithms of the fully functioning elastic net are incorporates herein by reference.
Crampin A C, Floyd S, Mwaungulu F, et al. Comparison of two versus three smears in identifying culture-positive tuberculosis patients in a rural African setting with high HIV prevalence. Int J Tuberc Lung Dis 2001; 5:994-9.
Hussain R, Kaleem A, Shahid F, et al. Cytokine profiles using whole-blood assays can discriminate between tuberculosis patients and healthy endemic controls in a BCG-vaccinated population. J Immunol Methods 2002; 264:95-108.
Franken K L, Hiemstra H S, van Meijgaarden K E, et al. Purification of his-tagged proteins by immobilized chelate affinity chromatography: the benefits from the use of organic solvent. Protein Expr Purif 2000; 18:95-9.
Benjamini Y, Hochberg Y. Controlling the False Discovery Rate—a Practical and Powerful Approach to Multiple Testing. J Roy Stat Soc B Met 1995; 57:289-300.
Joosten S A, Goeman J J, Sutherland J S, et al. Identification of biomarkers for tuberculosis disease using a novel dual-color RT-MLPA assay. Genes Immun 2012; 13:71-82.
Eldering E, Spek C A, Aberson H L, et al. Expression profiling via novel multiplex assay allows rapid assessment of gene regulation in defined signalling pathways. Nucleic Acids Res 2003; 31:e153.
Maertzdorf J, Ota M, Repsilber D, et al. Functional correlations of pathogenesis-driven gene expression signatures in tuberculosis. PLoS One 2011a; 6:e26938.
Maertzdorf J, Repsilber D, Parida S K, et al. Human gene expression profiles of susceptibility and resistance in tuberculosis. Genes Immun 2011b; 12:15-22.
Jacobsen M, Repsilber D, Gutschmidt A, et al. Candidate biomarkers for discrimination between infection and disease caused by Mycobacterium tuberculosis. J Mol Med (Berl) 2007; 85:613-21.
Cox J A, Lukande R L, Lucas S, Nelson A M, Van Marck E, Colebunders R. Autopsy causes of death in HIV-positive individuals in sub-Saharan Africa and correlation with clinical diagnoses. AIDS Rev 2010; 12:183-94.
Ansari N A, Kombe A H, Kenyon T A, et al. Pathology and causes of death in a group of 128 predominantly HIV-positive patients in Botswana, 1997-1998. Int J Tuberc Lung Dis 2002; 6:55-63.
Maertzdorf J, Weiner J, 3rd, Mollenkopf H J, et al. Common patterns and disease-related signatures in tuberculosis and sarcoidosis. Proc Natl Acad Sci USA 2012; 109:7853-8.
Robin X, Turck N, Hainard A, Tiberti N, Lisacek F, et al. (2011) pROC: an open-source package for R and S+ to analyze and compare ROC curves. BMC Bioinformatics 12:77.
Carpenter J, Bithell J (2000) Bootstrap confidence intervals: when, which, what? A practical guide for medical statisticians. Stat Med 19: 1141-1164.
Clopper C J, Pearson E S (1934) The use of confidence or fiducial limits illustrated in the case of the binomial. Biometrika 26: 404-413.
Altman D G, Bland J M (1994) Diagnostic tests 2: Predictive values. BMJ 309: 102.
Simel D L, Samsa G P, Matchar D B (1991) Likelihood ratios with confidence: sample size estimation for diagnostic test studies. J Clin Epidemiol 44: 763-770.

1. A method for detecting active tuberculosis (TB) in a subject derived sample in the presence of a complicating factor, comprising the step of detecting the modulation of at least 60% of the genes in a signature selected from the group consisting of:
a) a 27 gene signature shown in Table 3,
b) a 44 gene signature shown in Table 4,
c) a 53 gene signature shown in Table 5,
d) a combination of signatures a) and b), a) and c), b) and c) or a) and b) and c).

2. A method according to claim 1, wherein:
at least 80% of the genes of a given signature are detected; or
100% of the genes of a given signature are detected.

3. (canceled)

4. A method according to claim 1, wherein detection is performed employing a multiplex assay or wherein the gene signatures for use in the method are presented in the form of a microarray.

5. (canceled)

6. A method according to claim 1, wherein the detection method employs fluorescence or colorimetric analysis.

7. (canceled)

8. A method according to claim 1, wherein the complicating factor is:
latent TB;
the presence of a co-morbidity;
the presence of a co-morbidity selected from the group consisting of malignancy, HIV, malaria, pneumonia, Lower Respiratory Tract Infection, Pneumocystis Jirovecii Pneumonia, pelvic inflammatory disease, Urinary Tract Infection, bacterial or viral meningitis, hepatobiliary disease, cryptococcal meningitis, non-TB pleural effusion, empyema, gastroenteritis, peritonitis, gastric ulcer and gastritis;
HIV; or
malaria.

9-12. (canceled)

13. A method according to claim 1, wherein the patient derived sample is a body fluid sample, for example a blood or serum sample.

14. A method according to claim 1, wherein 6 genes in the 27 gene signature are up-regulated.

15. A method according to claim 14, wherein the remaining genes in the signature are down-regulated

16. A method according to claim 14, wherein the genes CD79A, CD79B, CXCR5, GNG7, CCR6 and 1ZNF296 are up-regulated.

17. A method according to claim 14, wherein genes C5, FAM20A, DUSP3, GAS6, S100A8, FCGR1B, LHFPL2, FCGR1A, MPO, FCGR1C, GAS6, C1QB, ANKRD22, FCGR1B, GBP6, C4ORF18, C1QC, FLVCR2, VAMP5, SMARCD3, LOC728744 are down-regulated.

18. A method according to claim 1, wherein 14 genes in the 44 gene signature are up-regulated.

19. A method according to claim 18, wherein the remaining genes are in the signature are down-regulated.

20. A method according to claim 18, wherein the genes ARG1, IMPA2, RP5-1022P6.2, ORM1, EBF1, PDK4, MAK, VPREB3, HS.131087, MAP7, TMCC1, HS.162734, MAP7, PGA5 are up-regulated.

21. A method according to claim 18, wherein the genes HM13BTN3A1, UGP2, CYB561, GBP6, CYB561, DUSP3, LOC196752, ALDH1A1, PRDM1, CERKL, HM13, RNF19A, MIR1974, PPPDE2, GJA9, CREB5, SERPING1, LOC389386, SEPT_{—}4, RBM12B, CALML4, LHFPL2, CASC1, C19ORF12, HLA-DPB1, CD74, ALDH1A1, AAK1, LOC100133800 are down-regulated.

22. A method according to claim 1, wherein the 16 genes in the 53 gene signature are up-regulated.

23. A method according to claim 22, wherein the remaining genes in the signature are down-regulated.

24. A method according to claim 22, wherein the genes GNG7, BLK, OSBPL10, CXCR5, HEY1, COL9A2, SPIB, LOC90925, ILMN_{—}1916292, EBF1, VPREB3, TMCC1, MAP7, PGA5, ILMN_{—}1893697 are up-regulated.

25. A method according to claim 22, wherein genes UGP2, BTN3A1, DUSP3, GBP6, CALML4, FZD2, CYB561, LHFPL2, CYB561, CASC1, RNU4ATAC, VPS13B, PPPDE2, ALDH1A1, GBP5, GAS6, SEP_{—}4, FCGR1B, POLB, CREB5, SIGLEC11, LOC389386, DEFA1B, LOC650546, FAM26F, FCGR1A, DEFA1B, ALDH1A1, ANKRD22, IFI27L2, DEFA1, MIR21, DEFA3, FCGR1C, UHMK1, CD74, IL15 and CREG1 are down-regulated.

26. A method according to claim 1, further comprising the steps of:
a. optionally normalising and/or scaling numeric values of the modulation
b. taking the normalised and/or scaled numeric values or the raw numeric values, each of which comprise both positive and/or negative numeric values and designating all said numeric values to be negative or alternatively all positive,
c. optionally refining the discriminatory power of one or more up-regulated genes and down-regulated genes by statistically weighting some of the numeric values associated therewith, and
d. summating the positive or negative numeric values obtained from step b) or step c) to provide a composite expression score,
wherein the composite expression score obtained from step d) is compared to a control and the comparison allows the sample to be designated as positive or negative for the relevant infection.

27. A gene chip comprising one or more of the gene signatures selected from the group consisting of:
a) 60 to 100% of a 27 gene signature shown in Table 3,
b) 60 to 100% of a 44 gene signature shown in Table 4,
c) 60 to 100% of a 53 gene signature shown in Table 5,
d) a combination of signatures a) and b), a) and c), b) and c) or a) and b) and c).

28-29. (canceled)
